(19) United States
(12) Patent Application Publication
(10) Pub. No.: US 2007/0167608 A1
(43) Pub. Date: Jul. 19, 2007

(54) Sugar chain structure profiling techniques

(76) Inventors: Jun Hirabayashi, Tsukuba-shi (JP); Atsushi Kuno, Tsukuba-shi (JP); Sachiko Nakamura, Tsukuba-shi (JP); Noboru Uchiyama, Tsukuba-shi (JP); Ken-ichi Kasai, Tsukui-gun (JP); Yoichiro Arata, Tsukui-gun (JP); Yoriko Takahashi, Tokyo (JP)

Correspondence Address: DARBY & DARBY P.C., P. O. BOX 5257, NEW YORK, NY, 10150-5257, US

(21) Appl. No.: 10/596,704

(22) PCT Filed: Jun. 30, 2004
(86) PCT No.: PCT/JP04/09600

§ 371 Date: Sep. 21, 2006

(30) Foreign Application Priority Data
Dec. 25, 2003 (JP) 2003-430615

(51) Int. Cl. C07K 14/00

(52) U.S. Cl. 530/350

(57) ABSTRACT

The present inventors discovered that lectins recognize extremely small differences in sugar chain structures, and that by using this ability to recognize sugar chain structures, numerous sugar chain structures can be distinguished using the strong or weak interaction patterns of about ten types of lectins. Therefore, by reference to and use of control interaction data, in which large amounts of data on the interactions between sugar chains and lectins have been collected, the structures of subject sugar chains can be identified, estimated, and such with high accuracy.

### TECHNICAL FIELD

The present invention relates to methods for rapidly and highly accurately identifying or estimating sugar chain structures.

### BACKGROUND ART

Examples of existing methods for analyzing sugar chain structures comprise NMR, methylation analysis, methods using a mass spectrometer, and methods that combine enzyme digestion and 2D mapping, however, each of these methods have their advantages and disadvantages. Although NMR is advantageous in that the absolute structure of a sugar chain can be determined, the sample required is very large, and both measurement and analysis take considerable time.

Although mass values can be obtained from analyses using a mass spectrometer, an inherent shortcoming is that the differences (distinctions between α and β, the types of monosaccharide, etc) between various structural isomers (anomers, epimers, diastereomers, linkage isomers, positional isomers) cannot be determined from mass alone.

Methods that combine enzyme digestion and 2D mapping are disadvantageous in that they are labor-intensive and time-consuming, since enzymes are not necessarily available for all sugar chain structures and analyses are performed using two columns. Also, these methods cannot distinguish between the structures of sugar chains which elute from two types of columns at the same or very close locations.

Methods for determining structure using enzymatic digestion require time-consuming enzyme reactions as well the repeated use of chromatography in procedures for analyses and recovery of the reaction products, and such methods are thus disadvantageous in that they require considerable labor and time.

Lectins have been known for more than 100 years as sugar-binding proteins, and they have been used to detect glycoproteins, sugar chains expressed on cells, and such. Frontal affinity chromatography (hereinafter referred to as FAC) is a method for quantitatively measuring interactions using the correlation between changes in the elution front, which occur when an affinity ligand of fixed concentration is continuously poured onto a column support on which analysis subjects are immobilized, and the strength of interaction with the ligand in the column.

By combining FAC with HPLC and then considerably downsizing, the present inventors were able to reduce the size of samples of ligands and analysis subjects, and substantially reduce measurement time, something previously difficult to achieve. In addition, using a fluorescence detector for detection not only considerably improved sensitivity but also enabled comprehensive analyses using pyridylaminated sugar chains (hereinafter referred to as PA sugar chains) including many commercially available products, making it possible for anyone to easily use multiple sugar chain libraries.

[Patent-Document 1] Japanese Patent Application Kokai Publication No. (JP-A) H08-201383 (unexamined, published Japanese patent application)

[Patent Document 2] Japanese Patent Application Kokai Publication No. (JP-A) H07-112996 (unexamined, published Japanese patent application)

[Patent-Document 3] Japanese Patent Application Kokai Publication No. (JP-A) 2001-13125 (unexamined, published Japanese patent application)

[Patent-Document 4] Japanese Patent Kohyo Publication No. (JP-A) 2002-544485 (unexamined Japanese national phase publication corresponding to a non-Japanese international publication)

[Non-Patent Document 1] Arata, Y., Hirabayashi, J., and Kasai, K., J. Chromatogr. A 890, 261-271, 2000

[Non-Patent Document 2] Arata, Y., Hirabayashi, J., and Kasai, K., J. Biol. Chem. 276, 3068-3077, 2001

[Non-Patent Document 3] Hirabayashi, J., Arata, Y., and Kasai, K., J. Chromatogr. A 905, 337-343, 2001

[Non-Patent Document 4] Hirabayashi, J., Hashidate, T., Arata, Y., Nishi, N., Nakamura, T., Hirashima, M., Urashima, T., Oka, T., Futai, M., Muller, W. E. G., Yagi, F., and Kasai, K., Biochim. Biophys. Acta 1572, 232-254, 2002

### DISCLOSURE OF THE INVENTION

A practical problem when analyzing sugar chain structures is the difficulty of comprehensive analysis, due to the variety of sugar chains. Also, sugar chain samples can often be obtained only in small amounts, and are hence expensive. Although sugar chain synthesis can be considered as a way of compensating for these problems, both chemical and biological synthesis methods are currently far from complete. Consequently, methods for rapidly and very accurately identifying and estimating sugar chain structures using extremely small amounts of sugar chains were required.

The present invention was achieved in view of the above circumstances, wherein the analysis of sugar chain structures has until now necessitated complex procedures and large amounts of samples. An objective of the present invention is to provide methods (hereinafter referred to as sugar chain structure profiling) for rapidly and very accurately identifying or estimating the structures of sugar chains using extremely small amounts of sugar chain samples, by comparing the characteristic interactions of control interaction data with the unique interactions of sugar chains, wherein interaction data is obtained using high-speed interaction analysis apparatus such as FAC apparatus, microarray scanner apparatus, or such.

The present inventors conducted dedicated research to solve the aforementioned problems. Specifically, the present inventors achieved the further downsizing of the aforementioned FAC system, the alignment of columns, and the complete automation of experimental procedures and data analyses, and thus succeeded in considerably improving throughput while maintaining the high level accuracy of FAC analysis. As a result, the present inventors discovered that lectin specificity differs between lectins more than previously known, and that due to their different affinities, each lectin recognizes extremely small differences in sugar chain structure. Thus, the present inventors judged that the wide differentiation abilities of each lectin, spanning high to low affinities, could be effectively used by comprehensively comparing quantitative data on their interactions with each sugar chain, and more specifically, by comparing intensity patterns of the affinity between each lectin and sugar chain, to mutually distinguish the structures of sugar chains in numbers far exceeding the number of lectins used, even when a relatively limited number of lectins (for example ten or more types) were used, as long as the specificities of the lectins were sufficiently different. Thus, by referring to and using control interaction data, which contains more data on the interactions between sugar chains and lectins, the structure of subject sugar chains can be much more easily identified and estimated than in the past, and in a highly accurate way. Moreover, these tools enable easy and highly accurate acquisition of information relating to the presence or absence of modified structures (such as α2-3 sialic acid, α2-6 sialic acid, α1-3 galactose, α1-6 fucose, bisect N-acetylglucosamine and such) characterizing each sugar chain structure.

Namely, the present invention provides the following (1) to (11), relating to methods or systems for profiling sugar chain structures:

(1) a method for analyzing a sugar chain structure, wherein the method comprises the steps of:

(a) introducing a fluorescence-labeled subject sugar chain to an FAC apparatus having parallel columns onto which each of a variety of proteins that interact with a sugar chain are immobilized; and

(b) measuring the interaction of the subject sugar chain with each of the proteins that interact with the sugar chain; wherein
when a combined pattern of a measured interaction of the subject sugar chain with each of the proteins that interact with the sugar chain matches a combined pattern of an interaction of a specific sugar chain with each of the proteins that interact with the sugar chain, taken from control data which comprise the interactions of a number of sugar chains with each of the proteins that interact with the sugar chain, the subject sugar chain is judged to have the same structure as the specific sugar chain;
   (2) the method of (1), wherein a protein that interacts with the sugar chain is a lectin, an enzymatic protein comprising a sugar-binding domain, a cytokine having affinity for a sugar chain, or an antibody that interacts with a sugar chain;
   (3) a system for analyzing a sugar chain structure using a computer and comprising:

(a) a storage means which stores data on the interaction of a number of sugar chains with a variety of proteins that interact with a sugar chain;

(b) a detection means which, when a fluorescence-labeled subject sugar chain is introduced into an FAC apparatus having parallel columns onto which each of the various proteins that interact with sugar chains is immobilized, detects the fluorescence intensity over time of a label attached to a subject sugar chain eluted from each column;

(c) a means for calculating data on the interaction of a subject sugar chain with each of the proteins that interact with sugar chains, based on an entered fluorescence intensity data, comparing a data combination of said interaction data with a data combination stored in (a), and selecting one or a number of sugar chains of known structure having a matching data combination pattern; and,

(d) a display means for displaying the selection results;

(4) the system of (3), wherein the arithmetic processing means of step (c) comprises the following (i) or (ii):

(i) a means for calculating the elution volume of a subject sugar chain from each column based on an entered fluorescence intensity data, calculating a difference between said elution volume and a control elution volume, comparing a data combination of said difference with a data combination stored in (a), and selecting one or a number of sugar chains of known structure having a matching data combination pattern; or,

(ii) a means for calculating the elution volume of a subject sugar chain from each column based on an entered fluorescence intensity data, calculating a difference between said elution volume and a control elution volume, calculating an affinity constant for the subject sugar chain with each of the proteins that interact with sugar chains based on said difference, comparing a data combination of said affinity constant with data combination stored in (a), and selecting one or a number of sugar chains of known structure having a matching data combination pattern;

(5) the system of (3) or (4), wherein a protein that interacts with a sugar chain is a lectin, an enzymatic protein having a sugar-binding domain, a cytokine having an affinity for a sugar chain, or an antibody that interacts with a sugar chain;

(6) a method for analyzing a sugar chain structure, wherein the method comprises:

(a) a step of contacting a fluorescence-labeled subject sugar chain with a substrate onto which each of a variety of proteins that interact with a sugar chain is immobilized; and

(b) a step of measuring the interaction of the subject sugar chain with each of the proteins that interact with the sugar chain by allowing an excitation light to act without carrying out a washing operation; wherein
when a combined pattern of a measured interaction of the subject sugar chain with each of the proteins that interact with the sugar chain matches a combined pattern of an interaction of a specific sugar chain with each of the proteins that interact with the sugar chain, taken from control data which comprise the interactions of a number of sugar chains with each of the proteins that interact with the sugar chain, the subject sugar chain is judged to have the same structure as the specific sugar chain;
   (7) the method of (6), wherein the excitation light is an evanescent wave;
   (8) the method of (6) or (7), wherein a protein that interacts with a sugar chain is a lectin, an enzymatic protein having a sugar-binding domain, a cytokine having an affinity for a sugar chain, or an antibody that interacts with a sugar chain;
   (9) a system for analyzing a sugar chain structure using a computer and comprising:

(a) a storage means which stores data on the interaction of a number of sugar chains with a variety of proteins that interact with a sugar chain;

(b) a detection means which, when a fluorescence-labeled subject sugar chain is contacted with a substrate onto which each of the various proteins that interact with sugar chains are immobilized, detects the intensity of an excited fluorescence after an incident excitation light has been shone on the substrate, without carrying out a washing procedure;

(c) a means for taking a data combination of the detected fluorescence intensity, comparing it with data stored in (a), and selecting one or a number of sugar chains of known structure having a matching data combination pattern; and

(d) a display means for displaying the selection results;

(10) the system of (9), wherein the excitation light is an evanescent wave; and

(11) the system of (9) or (10), wherein a protein that interacts with a sugar chain is a lectin, an enzymatic protein having a sugar-binding domain, a cytokine having an affinity for a sugar chain, or an antibody that interacts with a sugar chain.

The present invention provides novel methods for analyzing sugar chain structures. In the methods of the present invention, first, fluorescence-labeled subject sugar chains are introduced to an FAC apparatus having parallel columns onto which each of the various proteins that interact with the sugar chains are immobilized. Next, the interactions of the subject sugar chains with each of the proteins that interact with the sugar chains are measured. When a pattern of combinations of measured interactions of a subject sugar chain with each of the proteins that interact with the sugar chains matches a pattern of combinations of the interactions of a specific sugar chain with each of these proteins that interact with sugar chains, taken from the control data which contains data on the interactions of a number of sugar chains with each of the proteins that interact with the sugar chains, the subject sugar chain is judged to have a structure identical to that of the specific sugar chain. The methods of the present invention enable identification of the structure of a subject sugar chain when the subject sugar chain has a known structure. Even when the subject sugar chain has an unknown structure, a characteristic structure present in the subject sugar chain (such as α2-3 sialic acid, α2-6 sialic acid, α1-3 galactose, α1-6 fucose, and bisect N-acetylglucosamine) can be estimated, or similarities with a sugar chain of known structure can be indicated.

Examples of sugar chains in the present invention comprise glycoprotein-type sugar chains (N-linked sugar chains and O-linked sugar chains), glycolipid-type sugar chains, glycosaminoglycan-type sugar chains, and polysaccharide-derived oligosaccharide chains. In addition, 1) examples of N-linked sugar chains comprise high-mannose, hybrid, and complex N-linked sugar chains; 2) examples of O-linked sugar chains comprise mucin-type (O-GalNAc), O-Fuc-type, O-Man-type, and O-Glc-type O-linked sugar chains; 3) examples of glycolipid-type sugar chains comprise the ganglio-series, globo-series, the lacto-series, and the neolacto-series sugar chains; 4) examples of glycosaminoglycan-type sugar chains comprise hyaluronic acid, keratan sulfate, heparin, heparan sulfate, chondroitin sulfate, and dermatan sulfate; and 5) examples of polysaccharide-derived oligosaccharide chains comprise oligosaccharide chains and such derived from chitin, cellulose, curdlan, laminarin, dextran, starch, glycogen, arabinogalactan, alginic acid, fructan, fucoidan, and xylan.

In addition, examples of the sugar chains of the present invention comprise those used in the Examples, such as M3, M5A, hybrid (monoagalacto, bisect), NA1, NA1 (α1-6Fuc), NA2 (monoagalacto), NA2 (monoagalacto, bisect), NA2, NA2 (α1-6Fuc), A2, NA2 (bisect), NA3, NA3 (α1-6Fuc), NA4, NA4 (α1-6Fuc), NA5 (pentaagalacto, bisect), lactose, GA2, GA1, GM3-NeuAc, GM3-NeuGc, GM1, GM2, GD1a, GD1b, GD3, Gb3, Gb4, Forssman, LNnT, LNT, Galili pentasaccharide, B-hexasaccharide, LNFP-I, LNFP-II (Le^{a}), LNFP-III (Le^{x}), LNFP-II (Le^{b}), A-hexasaccharide, A-heptasaccharide, B-pentasaccharide, 6′-sialyl lactose, pLNH, βGalLac, βGal₂Lac, LN3, GN3, GN4, maltotriose, and sialyl Le^{x}.

The proteins of the present invention that interact with sugar chains also comprise peptides that interact with sugar chains. Examples of the proteins of the present invention that interact with sugar chains comprise lectins, enzymatic proteins comprising a sugar-binding domain, cytokines having an affinity for sugar chains, mutants thereof, and antibodies that interact with sugar chains.

Examples of the aforementioned lectins comprise lectins belonging to various molecular families obtained from animals, plants, fungi, bacteria, viruses, etc, and more specifically comprise “R-type lectins” related to the ricin B chain found in all organisms including bacteria; “calnexin-calreticulin” present in all eukaryotes and which is involved in the folding of glycoproteins; calcium-requiring “C-type lectins” widely found in multicellular animals and which comprise many typical lectins such as “selectins” and “collectins”; “galectins” which are widely distributed throughout the animal world and show specificity for galactose; “legume lectins” constituting a large family within the leguminous plants; “L-type lectins” structurally similar to the latter and involved in transport within animal cells; mannose-6-phosphate-binding “P-type lectins” involved in intracellular trafficking of lysosomal enzymes; “annexins” which bind to acidic sugar chains such as glycosaminoglycans; and “I-type lectins” which belong to the immunoglobulin superfamily and comprise “Siglec”.

In addition, examples of lectins of the present invention comprise those used in the Examples, such as ACA (Amaranthus caudatus agglutinin), BPL (Bauhinia purpurea lectin), ConA (Concanavalin A), DBA (Dolichos biflorus agglutinin), DSA (Datura stramonium agglutinin), ECA (Erythrina cristagalli agglutinin), EEL (Euonymus europaeus lectin), GNA (Galanthus nivalis agglutinin), GSL I (Griffonia simplicifolia lectin), GSL II (Griffonia simplicifolia lectin), HHL (Hippeastrum hybrid lectin), Jacalin (Jackfruit lectin), LBA (Lima bean agglutinin), LCA (Lens culinaris agglutinin), LEL (Loranthus europaeus lectin), LTL (Lotus tetragonolobus lectin), MPA (Maclura pomifera agglutinin), NPA (Narcissus pseudonarcissus agglutinin), PHA-E (Phytohemagglutinin), PHA-L (Phytohemagglutinin), PNA (Peanut agglutinin), PSA (Pisum sativum agglutinin), PTL-I (Psophocarpus tetragonolobus lectin), PTL-II (Psophocarpus tetragonolobus lectin), PWM (Pokeweed mitogen), RCA120 (Ricinus communis agglutinin), SBA (Soy bean agglutinin), SJA (Sophora japonica agglutinin), SNA (Sambucus nigra agglutinin), SSA (Sambucus sieboldiana agglutinin), STL (Solanum tuberosum lectin), TJA-I (Trichosanthes japonica agglutinin), TJA-II (Trichosanthes japonica agglutinin), UDA (Urtica dioica agglutinin), UEA I (Ulex europaeus agglutinin), VFA (Vicia faba agglutinin), VVA (Vicia villosa agglutinin), WFA (Wisteria floribunda agglutinin) and WGA (Wheat germ agglutinin).

Examples of the aforementioned enzymatic proteins comprising a sugar-binding domain comprise various types of glycosidases (xylanases, glucanases) and glycosyltransferases (UDP-GalNAc: polypeptide GalNAc transferases). In addition, examples of cytokines having an affinity for sugar chains comprise interleukin-2 (IL-2), interleukin-12 (IL-12), tumor necrosis factor α (TNF-α), and fibroblast growth factor (FGF). In addition, examples of antibodies interacting with sugar chains comprise antibodies against sugar chain-related tumor markers (CA19-9, Forssman antigen, T antigen, Tn antigen, and sialyl T antigen), blood type-related sugar chains (A, B, H, Lea, and Lex antigens), and differentiation-related antigens (Ii and SSEA-1-4).

In the methods of the present invention, various proteins that interact with sugar chains are each immobilized onto independent columns. Although the proteins to be immobilized that interact with sugar chains are at least one protein (and preferably at least two proteins) selected from all of the aforementioned proteins, the larger the number of proteins, the higher the precision and accuracy with which sugar chain structure can be estimated.

In addition, typical proteins that interact with sugar chains and which are thought to be effective in estimating the structure of a subject sugar chain can also be selected and used. In this case, time and labor can be saved even if the accuracy of sugar chain structure estimation is somewhat decreased.

Methods known among those skilled in the art can be used as methods for immobilizing the proteins onto the columns. For example, the methods described in the Examples can be used as a reference.

In the methods of the present invention, when a fluorescence-labeled subject sugar chain is introduced into an FAC apparatus having parallel columns onto which each of the various proteins that interact with sugar chains is immobilized, the fluorescence intensity of the label attached to the sugar chain is detected over a period of time as it elutes from each column.

Examples of fluorescence labeling agents in the present invention comprise 2-aminopyridine (2-AP), 2-aminobenzoic acid (2-AA), 2-aminobenzamide (2-AB), 2-aminoacridone (AMAC), p-aminobenzoic acid ethyl ester (ABEE), p-aminobenzonitrile (ABN), 2-amino-6-cyanoethylpyridine (ACP), 7-amino-4-methylcoumarine (AMC), 8-aminonaphthalene-1,3,6-trisulfate (ANTS), 7-aminonaphthalene-1,3-disulfide (ANDS), and 8-aminopyrene-1,3,6-trisulfate (APTS).

In the methods of the present invention, the interaction of a subject sugar chain with each of the proteins that interact with sugar chains is then calculated using a calculation method described below, based on information on the detected fluorescence intensity. Interactions between sugar chains and the proteins that interact with the sugar chains frequently have a dissociation constant (K_{d}) of 10⁻⁶ M or more, and are known to be generally weak interactions. It is known that by using an FAC apparatus, such weak interactions can be measured very accurately.

The principles of a method for measuring intermolecular interactions using an FAC apparatus are described below (FIG. 1).

When analyzing interactions using an FAC apparatus, a large amount of an analyte with a fixed concentration (the concentration is designated as [A]₀) is continuously injected into a small affinity column onto which one of the subjects of analysis is immobilized. At a certain point after the continuous injection, the column is no longer able to retain the analyte and elution of the analyte from the column begins. When the elution volume in the presence of an interaction between the immobilized ligands in the column and the analyte is designated as (V), and the elution volume in the absence of an interaction is designated as (V₀), a delay of (V-V₀) occurs in analyte elution, corresponding to the strength of the interaction. When the amount of analyte retained in the column is represented by [A]₀(V-V₀), the effective ligand concentration in the column is designated as Bₜ, and the dissociation constant is designated as K_{d}, then the below equation, which has the same form as the Michaelis-Menten equation of enzyme kinetics, is valid:

$$\begin{matrix}[ Equation 1 ] & \\ {[ A ]}_{o} ( V - {V}_{o} ) = \frac{{{B}_{t} [ A ]}_{0}}{{[ A ]}_{0} + {K}_{d}} \\ ( \begin{matrix}Corresponding to the & \\ Michaelis - Menten & v = \frac{{V}_{max} [ S ]}{[ S ] + {K}_{m}} \\ equation of enzyme kinetics & \end{matrix} ) \\ [ Equation 2 ] & ( Equation 1 ) \\ {K}_{d} = \frac{{B}_{t}}{V - {V}_{0}} - {[ A ]}_{0} IF {K}_{d} >> {[ A ]}_{0} then \\ {K}_{d} = \frac{{B}_{t}}{V - {V}_{0}} \\ [ Equation 3 ] & ( Equation 2 ) \\ {K}_{d} = \frac{1}{{K}_{a}} & ( Equation 3 )\end{matrix}$$

Since Bₜ is constant in a series of experiments that use the same column, it can be seen from Equation 2 that (V-V₀) is proportional to the strength of the interaction. If the Bₜ of the column used in the experiment is determined in advance, then the corresponding K_{d} can be calculated by simply determining (V-V₀) for each analyte. In addition, the dissociation constant (K_{d}) and the affinity constant (Kₐ) are related as shown in Equation 3.

Under conditions where the experimental system is completely ideal (conditions whereby the flow rate is sufficiently slow, equilibrium within the column is sufficiently established, and the interface in the flow path is undisturbed), the chromatogram should increase at once to the same concentration as the concentration of analyte continuously added to the column at the elution front (FIG. 2A). However, since elution in actual experiments is gradual due to the unevenness of flow path length resulting from diffusion or column width and such, a sigmoidal elution curve is depicted (FIG. 2B). If the elution curve has a sigmoid shape with perfect point symmetry, the elution front can be determined from the median of the sigmoid, however, in reality ideal point symmetry configuration seldom occurs. Therefore, when calculating the elution front (V), the area below the elution curve is calculated, and the elution front of an ideal elution with the same area is then calculated. Specifically, rectangular areas forming small strips (ΔSᵢ) are determined by multiplying data intervals (ΔV) integrated at fixed intervals with the signal intensity ([A]ᵢ) at that time point, and the area below the curve (ΣΔSᵢ) is determined by adding up these rectangular areas until an arbitrary measurement time (Vᵢ) (FIG. 2C). When considering a rectangle of height [A]₀ and area (ΣΔSᵢ) below the curve, the right-hand edge of the rectangle is the injected liquid amount Vᵢ, while the left-hand edge of the rectangle is the elution front (V) (FIG. 2D). V can be determined by Vᵢ-ΣΔSᵢ/[A]₀.

In the present invention, when a pattern of combinations of interactions (elution delay: V-V₀ value or Kₐ value) of a subject sugar chain with each of the proteins that interact with the sugar chains matches a pattern of combinations of the interactions of a specific sugar chain with each of these proteins, taken from among control data comprising data on the interactions of a number of sugar chains with each of the proteins that interact with the sugar chains, then the subject sugar chain is judged to have the same structure as said specific sugar chain.

The interactions of a number of sugar chains with the various proteins that interact with sugar chains in the control data are not limited to the V-V₀ values or Kₐ values obtained by the methods of the present invention. For example, values obtained by a method or system described below, or values obtained from various experimentation systems established thus far can also be used.

The aforementioned control data may be data that comprises the aforementioned interactions or data that comprises information on patterns of combinations of interactions. The patterns of combinations of interactions can be formed by a method described below. In addition, data stored in databases may also be used as the aforementioned control data. Also, a computer such as one described below can be used to judge whether combination patterns of interactions match.

The present invention also provides systems for analyzing sugar chain structures using a computer. These systems automatically display a calculation result when a fluorescence-labeled subject sugar chain is introduced into an FAC apparatus having parallel columns onto which each of the various proteins that interact with the sugar chains are immobilized. Mass spectrometry or enzyme digestion can also be combined with the systems of the present invention, and since even more reliable data can be obtained using these methods, such systems are very useful.

An example of a composition of the systems of the present invention is shown in FIG. 3. A system of the present invention is composed of the following:
(a) a storage means (database) which stores data on the interaction of a number of sugar chains with a variety of proteins that interact with a sugar chain;
(b) a detection means which, when a fluorescence-labeled subject sugar chain is introduced into an FAC apparatus having parallel columns onto which each of the various proteins that interact with sugar chains is immobilized, detects the fluorescence intensity over time of a label attached to a subject sugar chain eluted from each column;
(c) a computer comprising a means for calculating data on the interaction of a subject sugar chain with each of the proteins that interact with sugar chains, based on an entered fluorescence intensity data, comparing a data combination of said interaction data with a data combination stored in (a), and selecting one or a number of sugar chains of known structure having a matching data combination pattern; and,
(d) a display means for displaying the selection results.

Both the case of a database set outside the computer, as in FIG. 3, and the case of a database set within the computer, as in FIG. 4, are allowed.

An example of a composition of a computer in the system of the present invention is shown in FIG. 4. Input Means 1 and Output Means 2 are connected to Bus Line 3. Temporary Storage Means 4 temporarily stores the entered data, the calculated data, and such. Central Processing Unit (CPU) 5 carries out various operations upon receiving commands from the programs of the present invention. Data on the interaction of a number of sugar chains with various proteins that interact with the sugar chains and/or data on patterns of combinations of this interaction data are stored in Storage Means (Database) 7. The interaction data are not limited to the V-V₀ or Kₐ values obtained by a method or system that uses the FAC apparatus of the present invention, or to fluorescence intensity data obtained by a method or system that uses the microarray scanner apparatus described below, and information obtained from various experimental systems established thus far can be used.

Various types of programs, comprising programs for executing the processes of the present invention, are stored in Storage Means 6. The programs for executing the processes of the present invention at least comprise Program 61, which calculates data on the interactions of a subject sugar chain with each of the proteins that interact with sugar chains based on the entered fluorescence intensity data; Program 62 which takes a data combination on the interactions of a subject sugar chain with each of the proteins that interact with sugar chains, and compares this data with data combinations stored in databases of data on the interactions of a number of sugar chains with various proteins that interact with the sugar chains, and selects one or a number of sugar chains of known structure (data stored in the database of sugar chains of known structure) with matching patterns of data combination; the Display Program 63; and Program 64 to control the above. In addition, the programs may also comprise programs for executing the processes for a system that uses a microarray scanner apparatus described below. Such computers can be used not only in systems that use FAC apparatus, but also in systems that use microarray scanner apparatus.

Instead of storing Program 62 (or as well as storing Program 62), Storage Means 6 may store Program 62-1, which generates patterns from combinations of data on the interaction of subject sugar chains with each of the proteins that interact with sugar chains; Program 62-2, which generates patterns from combinations of data stored in a database of data on the interactions of a number of sugar chains with various proteins that interact with sugar chains; and Program 62-3, which takes the patterns from the combinations of data on the interactions of subject sugar chains with each of the proteins that interact with sugar chains, and compares it with patterns stored in a database of combinations of data on the interactions of a number of sugar chains with various proteins that interact with sugar chains, and selects one or a number of sugar chains of known structure with a matching pattern of combined data.

Program 61 is composed of Program 61-1, which calculates the elution volume of a subject sugar chain from parallel columns onto which each of the various proteins that interact with sugar chains are immobilized (Arata, Y, Hirabayashi, J., and Kasai, K., J. Chromatogr. 905, 337-343, 2001; Arata, Y, Hirabayashi, J. and Kasai, K., J. Biol. Chem. 276, 3068-3077, 2001); and Program 61-2, which based on said elution volume calculates data on the interactions of a subject sugar chain with each of the proteins that interact with sugar chains.

By using Program 61-1, complex calculations can be automatically executed using common spreadsheet software. An actual example of a spreadsheet is shown below (FIG. 5).

Column B and Column D: Time and voltage data output from the FAC system are directly pasted here.

Column C: Displays the elution volume from the time data of column B and the flow rate (here, ΔV is 0.002084 mL).

Column E: Since the voltage at the time of data import is not zero, the average voltage value is determined (shown in D21) for ten points immediately after the start of data import (the location of these ten points can be set) and zero point correction is carried out by subtracting this value from the raw voltage value data (column D) to obtain the value [A]ᵢ.

Column A: Column used for plateau judgment

A plateau is judged to be reached when the column E voltage values of the ten previous points differ by ±1% or less, and this state continues over five consecutive points (the segment over which plateau judgment is performed can be set).

Column F: The column E voltage value at the point of reaching plateau is set as 100, and the column E voltage values of each data point are shown as a percentage.

Column G: The areas of small rectangular strips (ΔSᵢ) are calculated for each data point by multiplying [A]ᵢ of column E and ΔV.

Column H: ΔΣSᵢ is calculated by cumulatively adding the ΔSᵢ values determined in column G.

Column I: ΣΔSᵢ/[A]₀ is calculated by dividing the value of ΣΔSᵢ in column H by the voltage value [A]₀ at the point of reaching plateau (shown in D18).

Column J: The value of V is calculated by subtracting ΣΔSᵢ/[A]₀ calculated in column I from Vᵢ of column C. This value converges to a constant value when elution reaches a plateau and the voltage value becomes constant. The converged value of V at the point of reaching plateau is used as the elution volume.

In addition, Program 61-2 is either 1) a program that calculates the difference between the elution volume obtained by executing Program 61-1 and a control elution volume (V-V₀ value); or 2) a program that calculates the difference between the elution volume obtained by executing the Program 61-1 and a control elution volume (V-V₀), and further calculates, based on said difference, affinity constants (Kₐ) for a subject sugar chain and each of the proteins that interact with the sugar chains, using an aforementioned calculation formula.

Herein, the control elution volume refers to the elution volume (V₀) of a fluorescence-labeled analyte that does not interact with the various proteins that interact with sugar chains, which are immobilized in the column. Although analytes can be suitably selected by one skilled in the art, rhamnose is used as an analyte when, for example, galectin is used as a protein that interacts with sugar chains.

In addition, Program 62 is a program that compares data combinations of the interaction data (V-V₀ or Kₐ values) obtained by executing Program 61 with data combinations of the data stored in databases on the interaction of a number of sugar chains with various proteins that interact with sugar chains, and selects one or a number of sugar chains of known structure and matching combined data patterns.

When collating data combinations of interaction data, the values of the data combinations of interaction data may be compared. A function may be incorporated in Program 62 that compares, for example, the values of combinations of V-V₀ or Kₐ data obtained by executing Program 61 with the values of combinations of V-V₀ or Kₐ data stored in a database, and then selects one or a number of sugar chains of known structure, based on the closeness of these values. In addition, in the process of comparing data combinations of the interaction data, the data may be set into patterns, and these patterns may be compared. In view of this, Programs 62-1 to 62-3 may be stored in Storage Means 6 instead of Program 62 (or together with Program 62).

Program 62-1 is a program that generates patterns from data combinations of interaction data (V-V₀ or Kₐ values) obtained by executing Program 61. In addition, Program 62-2 is a program that generates patterns from data combinations stored in databases of data of the interactions of a number of sugar chains with various proteins that interact with sugar chains. When generating these patterns, interaction data can be standardized using a suitable internal standard. For example, data on each interaction can be standardized by converting data on the interactions of a subject sugar chain with each of the proteins that interact with sugar chains, and data stored in a database of the interactions of a number of sugar chains with various proteins that interact with sugar chains, into relative values with respect to data on the interactions of a reference sugar chain. Specifically, Program 62-1 comprises a program that converts data on the interactions of a subject sugar chain with each of the proteins that interact with the sugar chain into relative values with respect to data on the interactions of a reference sugar chain, while Program 62-2 comprises a program that converts data stored in a database of the interactions of a number of sugar chains with various proteins that interact with sugar chains into relative values with respect to data on the interactions of a reference sugar chain. For example, a V-V₀ value can be converted to a relative value using Equation 4 below, while a Kₐ value can be converted to a relative value using Equation 5 below (conversion of data stored in a database of the interactions of a number of sugar chains with various proteins that interact with sugar chains into relative values can also be carried out in the same manner). An example of a reference sugar chain is a sugar chain for which the V-V₀ value is in the range of 10 μL to 20 μL, however, the V-V₀ value of a reference sugar chain in the present invention is not limited to the range of 10 μL to 20 μL and can be set to an arbitrary range or value.

[Equation 4]
Relative value of the V-V₀ value of a subject sugar chain=(V-V₀ of a subject sugar chain/V-V₀ of a reference sugar chain)×100 (Equation 4)
[Equation 5]
Relative value of the Kₐ value of a subject sugar chain=Kₐ of a subject sugar chain/Kₐ of a reference sugar chain)×100 (Equation 5)

When V-V₀ or Kₐ are negative, the relative value is calculated with V-V₀ and Kₐ considered as zero.

A function is incorporated in Program 62-1 and Program 62-2 such that, for example, entering an arbitrary threshold value divides the interaction data into levels within the range of this threshold and encodes them (applies, for example, a different number or a different color to each level).

Program 62-3 is a program that compares patterns obtained by executing Program 62-1 with patterns obtained by executing Program 62-2, and selects one or a number of sugar chains of known structure and matching patterns. For example, the multivariate distance between two points can be determined for a pattern of combinations of data of relative data on the interactions of a subject sugar chain with each of the proteins that interact with the sugar chain, and a pattern of combinations of data stored in a database of relative data on the interaction of a number of sugar chains with various proteins that interact with sugar chains. Based on this distance, a model (group) with a low degree of difference in the patterns (a model (group) with high similarity) can be selected. Namely, Program 62-3 comprises a program that takes patterns of combinations of relative data on the interaction of a subject sugar chain with each of the proteins that interact with the sugar chain, and compares them with patterns of combinations of data stored in a database of relative data on the interactions of a number of sugar chains with various proteins that interact with sugar chains, and then selects one or a number of sugar chains of known structure and a pattern of combination data that matches (specifically, selects one or a number of sugar chains of known structure that rank high in order of pattern similarity). In calculating the degree of variance (or degree of similarity), the Manhattan distance, for example, can be used as the distance scale. The multivariate distance d_{ab} between sugar chain a and sugar chain b can be calculated using Equation 6 below from the difference in the m variable of each. The symbols used in Equation 6 are as follows: a: subject sugar chain, b: sugar chain of known structure, j: protein interacting with sugar chains, m: number of proteins interacting with sugar chains.

$$[ Equation 6 ]$$

$${d}_{ab} = {\sum }_{j}^{m} \vert {x}_{aj} - {x}_{bj} \vert \qquad ( Equation 6 )$$

When patterns of data are stored in a database, Program 62-3 compares the patterns obtained by executing Program 62-1 with the patterns stored in the database, and selects one or a number of sugar chains of known structure and matching pattern. Program 62-3 incorporates a function that, for example, compares the codes of sugar chains of known structure with the code of a subject sugar chain, and selects a sugar chain of known structure which has a code that matches that of the subject sugar chain.

Program 63 displays, for example, a list of chromatograms, interaction data, selected sugar chains of known structure, etc.

In the present invention, the aforementioned programs can also be integrated into a single program.

The following provides an example of a flow of processes executed by the systems of the present invention: First, when a fluorescence-labeled subject sugar chain is introduced into an FAC apparatus having parallel columns onto which each of the various proteins that interact with sugar chains are immobilized, the fluorescence intensity of the label attached to the subject sugar chain eluted from each column is detected over a period of time. Fluorescence intensity data are then automatically entered into a computer. The entered data can be stored in a storage means or temporary storage means of the computer.

In the present invention, an arithmetic processing means such as a Central Processing Unit (CPU) can receive a command from Program 63 in the storage means, read the fluorescence intensity data stored in the storage means or temporary storage means, and display said fluorescence intensity data in the form of, for example, a chromatogram.

As an example of a processing flow, data on the interaction of the subject sugar chain with each of the proteins that interact with sugar chains is then calculated based on the entered fluorescence intensity data. Usually, in this processing step, the arithmetic processing means such as a Central Processing Unit (CPU) normally receives a command from Program 61 in the storage means, reads the fluorescence intensity data stored in the storage means or temporary storage means, and calculates the interaction data. The calculated interaction data may be stored in the storage means or the temporary storage means of the computer. In addition, the calculated interaction data may also be stored in a database. By accumulating calculated interaction data, a very practical database, of a scale larger than seen before, of data on the interactions of sugar chains with proteins that interact with sugar chains can be constructed.

In the present invention, an arithmetic processing means such as a Central Processing Unit (CPU) can also receive commands from Program 63 in the storage means, read the interaction data stored in the storage means or temporary storage means, and display said interaction data.

As an example of a processing flow, combinations of data on the interactions of a subject sugar chain with each of the proteins that interact with sugar chains are then compared with combinations of data stored in a database of data on the interactions of a number of sugar chains with various proteins that interact with sugar chains, and one or a number of sugar chains of known structure and a matching pattern of combination data are selected. In this processing step, an arithmetic processing means such as a Central Processing Unit (CPU) receives a command from Program 62 in the storage means, reads the data combinations of interaction data stored in the storage means or temporary storage means, and the data combinations of data stored in a database of the interactions of a number of sugar chains with various proteins that interact with sugar chains, then compares each combination of data, and selects one or a number of sugar chains of known structure and a matching pattern of combination data. Data on the selected sugar chain of known structure can be stored in the storage means or temporary storage means of the computer.

When the database is outside the computer, an arithmetic processing means such as a Central Processing Unit (CPU) receives a command from Program 62 in the storage means, enters data combinations of the data stored in a database of interactions of a number of sugar chains with various proteins that interact with sugar chains, reads the data combinations of the interaction data stored in the storage means or temporary storage means, compares each combinations of data, and selects one or a number of sugar chains of known structure and a matching pattern of combination data.

The processes are carried out with a similar flow when using Programs 62-1 to 62-3 instead of Program 62.

As an example of a processing flow, the selection result is then displayed by the display means. In this processing step, an arithmetic processing means such as a Central Processing Unit (CPU) receives a command from Program 63 in the storage means, reads the above-described data on the sugar chain of known structure, which is stored in the storage means or temporary storage means, and displays this data.

Moreover, the present invention provides methods for analyzing sugar chain structures that use microarray scanner apparatus. In the present invention, since various proteins that interact with sugar chains are immobilized onto a substrate, a number of interactions can be observed at one time. Specifically, since interactions between sugar chains and various proteins that interact with sugar chains can be simultaneously observed using the methods of the present invention, higher throughput profiling becomes possible.

Examples of the sugar chains and the proteins that interact with sugar chains of the present invention comprise the above-described sugar chains and proteins that interact with sugar chains. In addition, examples of the substrates of the present invention comprise glass, quartz glass, synthetic quartz glass, but are not limited thereto. Moreover, the substrates onto which the various proteins that interact with sugar chains are immobilized are preferably substrates coated with compounds that comprise an epoxy group as the active group, and onto which various proteins that interact with sugar chains are immobilized.

A preferable but non-limiting example of a compound that comprises an epoxy group as the active group is 3-glycidoxypropyl trimethoxysilane (GTMS). Other examples comprise 2-(3,4epoxycyclohexyl)ethyltrimethoxysilane, 3-glycidoxypropylmethyldiethoxysilane, 3-glycidoxypropyltriethoxysilane, or silane coupling compounds comprising a number of epoxy groups at the end of a branched spacer, preferably further comprising polyethylene glycol, proteins, biotin/avidin, and such as a spacer.

Substrates onto which various proteins that interact with sugar chains are immobilized can be produced using the method described below.

First, a compound comprising an epoxy group as the active group is coated onto the substrate. For example, when GTMS is used as the compound comprising an epoxy group as the active group, coating can be carried out using the method described in the Examples. Specifically, the glass surface is treated by immersing a slide glass in a 10% KOH/MeOH solution and allowing it to stand for one hour while the container is shaken. After washing with a sufficient amount of purified water (MilliQ water), the slide glass is dried in an oven at 60° C. Next, the slide glass is immersed in a 2% GTMS acetone solution and allowed to react in the dark for one hour while the container is shaken. The alkoxysilyl groups of GTMS are hydrolyzed by water and become silanol groups. Since these silanol groups are unstable, they partially condense over time and form oligomers that subsequently attach to the glass surface via hydrogen bonding. After the reaction, the slide glass is dried for eight hours in an oven at 110° C. The drying treatment causes a dehydration-condensation reaction with the silanol groups on the glass surface, resulting in strong covalent bonding. The sequence of a GTMS coating method is shown in FIG. 12.

Next, the proteins that interact with sugar chains are immobilized onto the substrate, which is coated with a compound that comprises an epoxy group as the active group. Specifically, immobilization can be performed by spotting compounds which comprise amino groups as active groups onto said substrate and allowing reaction. STANPMAN, from Nippon Laser Electronics Ltd., or such can be used as the spotter. When a compound that comprises an amino group as the active group is a lectin, the concentration of the spotted lectin is preferably 1 mg/mL or more. Further more preferably, unbound lectin can be removed after the spotting treatment by washing using PBS comprising Tween20 (PBST).

The aforementioned substrates onto which the proteins that interact with sugar chains are immobilized are preferably substrates that constitute a number of reaction vessels. More preferably, they are substrates that constitute a number of reaction vessels by affixing a rubber having a number of holes. As an example of this, eight reaction vessels are produced by affixing an 8-hole rubber, designed and developed by the present inventors, to a given position on a slide glass after lectin immobilization, as described in the Examples. This 8-hole rubber has eight rectangular holes in an orderly arrangement and can form eight reaction vessels when affixed to a slide glass. Filling these reaction vessels with a fluorescence-labeled probe solution enables smooth contact with the proteins that interact with sugar chains. In addition, these reaction vessels are not limited to 8-hole rubbers and, for example, reaction areas can also be formed by coating non-spotting areas of the glass surface with water repellants. More preferably, a large number of reaction areas are formed.

In the methods of the present invention, a fluorescence-labeled subject sugar chain is contacted with a substrate, onto which substrate the aforementioned various proteins that interact with sugar chains are immobilized.

In the present invention, examples of fluorescence labeling agents for the subject sugar chains comprise 2-aminopyridine, Cy3, Cy3.5, Cy5, tetramethyl rhodamine, various types of fluorescent dyes comprising a fluorescein backbone, the Alexa series of fluorescent dyes manufactured by Molecular Probes Inc., and quantum dot fluorescent dyes, but are not limited thereto provided that the substance has the property of fluorescently labeling a sugar chain.

In the methods of the present invention, without washing the substrate, the interactions of a subject sugar chain with each of the proteins that interact with sugar chains are then measured using an excitation light.

Since the interactions between sugar chains and the proteins that interact with sugar chains are weak compared to generally well-known protein-protein interactions, there were cases in which a dissociation reaction proceeded between the sugar chains and the proteins that interact with sugar chains as a result of removing the probe solution and the washing operations, and in these cases accurate interaction data under equilibrium conditions could not be obtained.

The present inventors solved the above problem by using an excitation light to measure the intensity of the excited fluorescence, without washing the probe solution. More specifically, this measurement method involves shining an incident excitation light from the substrate side without immobilization, and detecting the excited fluorescence. There is no particular limitation as to the excitation lights of the present invention, and examples comprise a light source spliced from white light, preferably a laser light comprising a single wavelength, and more preferably an evanescent wave. Although an evanescent-type excitation microarray scanner is preferably used to detect the excitation light, a confocal-type microarray scanner can also be used.

For example, when excitation light in an evanescent excitation system is totally internally reflected, a faint light referred to as “evanescent light” permeates from the glass interface at a height of 200 nm to 300 nm (about half the excitation wavelength). When using this evanescent light to excite a fluorescent substance, a solution containing probe molecules is contacted to the top of a slide glass, and fluorescence is observed using an incident excitation light, in which case the probe molecules involved in binding reactions can be observed fluorescently with hardly any excitement of those probe molecules engaged in Brownian motion.

In the present invention, when a measured pattern of combinations of interactions (fluorescence intensity values) of a subject sugar chain with each of the proteins that interact with sugar chains matches a pattern of combinations of interactions of a specific sugar chain with each of the proteins that interact with sugar chains, taken from control data that comprises the interactions of a number of sugar chains with each of the proteins that interact with sugar chains, the subject sugar chain is judged to have the same structure as said specific sugar chain. By using the methods of the present invention, the structure of a subject sugar chain can be identified when the structure of the subject sugar chain is known. Even when the structure of the sugar chain is unknown, a characteristic structure present in the subject sugar chain (such as α2-3 sialic acid, α2-6 sialic acid, α1-3 galactose, α1-6 fucose, or bisect N-acetylglucosamine) can be predicted or a similarity with sugar chains of known structure can be pointed out.

Fluorescence intensity values obtained by the methods or below-described systems of the present invention, V-V₀ values or Kₐ values obtained by the aforementioned methods or systems, or those obtained from various previously established experimental systems can be used as the control data interactions of a number of sugar chains with various proteins that interact with sugar chains.

The aforementioned control data may be data comprising the aforementioned interactions, or may be data comprising patterns of combinations of interactions. Patterns of combinations of interactions can be formed by a method described below. In addition, data stored in a database can also be used as the aforementioned control data. Moreover, the computer described below can be used to judge whether or not patterns of combinations of interactions match.

Furthermore, the present invention also provides systems that use computers to analyze sugar chain structures. These systems automatically display the structure of a subject sugar chain upon placing a substrate in a microarray scanner apparatus, wherein each of the various proteins that interact with sugar chains and which have been contacted with a fluorescence-labeled subject sugar chain have been immobilized on to the substrate. In the systems of the present invention, the step of contacting a fluorescence-labeled subject sugar chain with a substrate, onto which each of the various proteins that interact with sugar chains have been immobilized, can also be automated. Specifically, by guiding a micro flow path system into the reaction vessels on the substrate, and controlling the type, concentration, and flow rate of the solutions sent into the flow path, the steps of blocking and removing the blocking solution, and the step of contacting the fluorescence-labeled sugar chain solution can be controlled in one dimension. Mass spectrometry or enzyme digestion can also be combined with the systems of the present invention, which is very useful since use of these methods enables data with even greater reliability to be obtained.

An example of a composition of the system of the present invention is shown in FIG. 3. A system that uses a microarray scanner apparatus is composed of the following:
(a) a storage means (database) which stores data on the interaction of a number of sugar chains with a variety of proteins that interact with a sugar chain;
(b) a detection means which, when a fluorescence-labeled subject sugar chain is contacted with a substrate onto which each of the various proteins that interact with sugar chains are immobilized, detects the intensity of an excited fluorescence after an incident excitation light has been shone on the substrate, without carrying out a washing procedure;
(c) a computer comprising an arithmetic processing means for taking a data combination of the detected fluorescence intensity, comparing it with data stored in (a), and selecting one or a number of sugar chains of known structure having a matching data combination pattern; and
(d) a display means for displaying the selection results.

A database is allowed, both when it is outside the computer, as in FIG. 3, and when it is within the computer, as in FIG. 4.

An example of a composition of a computer in a system of the present invention is shown in FIG. 4. Input Means 1 and Output Means 2 are connected to Bus Line 3. Temporary Storage Means 4 temporarily stores the entered data, the calculated data, and such. The Central Processing Unit (CPU) 5 performs various arithmetic operations after receiving a command from a program of the present invention. Data on the interactions of a number of sugar chains with various proteins that interact with sugar chains and/or data on the patterns of combinations of said interaction data are stored in Storage Means (database) 7. Fluorescence intensity data obtained by the methods or systems of the present invention that use a microarray scanner apparatus, V-V₀ or Kₐ values obtained by the aforementioned methods or systems, or data obtained from various experimental systems established to date can be used as the interaction data.

Various types of programs, comprising the programs for executing the processes of the present invention, are stored in Storage Means 6. Programs for executing the processes of the present invention comprise at least: Program 61, which takes combinations of data on the entered fluorescence intensity, and compares them with combinations of data stored in a database on the interactions of a number of sugar chains with various proteins that interact with sugar chains, and then selects one or a number of sugar chains of known structure that have a matching pattern of combination data; the display Program 62; and Program 63 for control thereof. In addition, a program for executing processes in a system that uses an FAC apparatus may also be comprised. Such computer can be used not only in systems that use a microarray scanner apparatus, but also in systems that use an FAC apparatus.

In the process of comparing the data combinations of interaction data, the values of data combinations of interaction data may also be compared. A function may be incorporated in Program 61, which, for example, compares the values of combinations of data on the entered fluorescence intensity with values stored in a database of data combinations of interaction data and selects one or a number of sugar chains of known structure based on the closeness to these values.

In addition, in the process of comparing the data combinations of interaction data, data combinations of interaction data may be formed into patterns and these patterns may also be compared. In view of this, instead of Program 61 (or together with Program 61), Storage Means 6 may store: Program 61-1, which generates patterns from data combinations of entered fluorescence intensities; Program 61-2, which generates patterns from data combinations of data stored in a database of interactions of a number of sugar chains with various proteins that interact with sugar chains; and Program 61-3, which compares the patterns of data combinations of entered fluorescence intensities with the patterns of data combinations of data stored in a database of interactions of a number of sugar chains with various proteins that interact with sugar chains, and selects one or a number of sugar chains of known structure that have a matching combination pattern. When generating patterns, interaction data can be standardized using a suitable internal standard. For example, data on each interaction can be standardized by converting the entered fluorescence intensity data and the data stored in a database of interactions of a number of sugar chains with various proteins that interact with sugar chains into relative values with respect to the fluorescence intensity data of a reference sugar chain. Specifically, Program 61-1 comprises a program that converts the entered data on fluorescence intensity into relative values with respect to the fluorescence intensity data of a reference sugar chain, while Program 61-2 comprises a program that converts the data stored in a database of interactions of a number of sugar chains with various proteins that interact with sugar chains into relative values with respect to data on the fluorescence intensity of a reference sugar chain. For example, fluorescence intensity data can be converted into relative values using Equation 7 below (conversion of the data stored in a database of interactions of a number of sugar chains with various proteins that interact with sugar chains into relative values can also be carried out in the same manner). A sugar chain whose properties have already been sufficiently investigated can be used as the reference sugar chain.

[Equation 7]
Relative value of fluorescence intensity data of a subject sugar chain=(fluorescence intensity data of a subject sugar chain/fluorescence intensity data of a reference sugar chain)×100 (Equation 7)

When the fluorescence intensity data is negative, the relative value is calculated with the fluorescence intensity data considered as zero.

A function is incorporated in Program 61-1 and Program 61-2 such that, for example, entering an arbitrary threshold value divides the interaction data into levels within the range of this threshold and encodes them (applies, for example, a different number or a different color to each level).

Program 61-3 is a program that compares patterns obtained by executing Program 61-1 with patterns obtained by executing Program 61-2, and then selects one or a number of sugar chains of known structure that have a matching pattern of data combination. For example, the multivariate distance between two points can be determined for a pattern of combinations of data of relative data on the value of entered fluorescence intensity and a pattern of combinations of data stored in a database of relative data on the interactions of a number of sugar chains with various proteins that interact with sugar chains. Based on this distance, a model (group) with a low degree of pattern variance (a model (group) with a high degree of pattern similarity) can be selected. Namely, Program 61-3 comprises a program that takes patterns of combinations of relative data on the entered fluorescence intensity data, and compares them with patterns of combinations of data stored in a database of relative data on the interactions of a number of sugar chains with various proteins that interact with sugar chains, and then selects one or a number of sugar chains of known structure and with a pattern of combination data pattern that matches (specifically, selects one or a number of sugar chains of known structure that rank high in order of pattern similarity). In calculating the degree of variance (or degree of similarity), the Manhattan distance, for example, can be used as the distance scale. The multivariate distance d_{ab} between sugar chain a and sugar chain b can be calculated with the aforementioned Equation 6 from the difference in the m variable of each.

When patterns of data are stored in a database, Program 61-3 compares the patterns obtained by executing Program 61-1 with the patterns stored in the database, and selects one or a number of sugar chains of known structure and matching pattern. Program 61-3 incorporates a function that, for example, compares the codes of sugar chains of known structure with the code of a subject sugar chain, and selects a sugar chain of known structure that has a code that matches that of the subject sugar chain.

Program 62 displays, for example, the fluorescence intensity data, interaction data, selected sugar chains of known structure, or such.

In the present invention, the aforementioned programs can also be integrated into a single program.

The following provides an example of a flow of processes executed by the systems of the present invention: First, when substrates, onto which are immobilized each of the various proteins that interact with sugar chains and which were contacted with a fluorescence-labeled subject sugar chain, are placed in a microarray scanner apparatus, an incident excitation light is shone onto said substrates and the intensity of the excited fluorescence is detected. When a number of substrates are placed in the microarray scanner apparatus, the number of substrates are sequentially and automatically fixed in the detection unit and scanned. As an example of a processing flow, the fluorescence intensity data is then automatically entered into a computer. The entered data can be stored in the storage means or temporary storage means of the computer. In addition, fluorescence intensity data may also be stored in a database. By accumulating fluorescence intensity data, a very practical database, of a scale larger than seen before, of data on the interactions of sugar chains with proteins that interact with sugar chains can be constructed.

In the present invention, an arithmetic processing means such as the Central Processing Unit (CPU) can receive a command from Program 62 in the storage means, read the fluorescence intensity data stored in the storage means or temporary storage means, and display said fluorescence intensity data. For example, by taking as standard the fluorescence intensity emitted by a spot of a sample protein that interacts with sugar chains whose properties have already been sufficiently investigated (an internal standard spot), the values of each spot for which the fluorescence value has been adjusted can be displayed. A number of internal standard spots may also be used.

As an example of a processing flow, combined data on the entered fluorescence intensity is then compared with data combinations of data stored in a database of the interactions of a number of sugar chains with various proteins that interact with sugar chains, and one or a number of sugar chains of known structure having a matching combination data pattern are selected. In this processing step, an arithmetic processing means such as the Central Processing Unit (CPU) receives a command from Program 61 in the storage means, reads the data combinations of fluorescence intensities stored in the storage means or temporary storage means and the data combinations of the data stored in a database of interactions of a number of sugar chains with various proteins that interact with sugar chains, compares each of the data combinations, and selects one or a number of sugar chains of known structure having a matching data combination pattern. Data on the selected sugar chain of known structure can be stored in the storage means or temporary storage means of the computer.

When the database is outside of the computer, an arithmetic processing means such as the Central Processing Unit (CPU) receives a command from Program 61 in the storage means, enters the data combinations of data stored in a database of interactions of a number of sugar chains with various proteins that interact with sugar chains, reads the data combinations of fluorescence intensities stored in the storage means or temporary storage means, compares each data combination, and selects one or a number of sugar chains of known structure which have a matching pattern of data combination.

Processes are carried out with a similar flow when using Programs 62-1 to 62-3 instead of Program 61.

As an example of a processing flow, the selection result is then displayed by the display means. In this processing step, an arithmetic processing means such as the Central Processing Unit (CPU) receives a command from Program 62 in the storage means, reads the known structure sugar chain data stored in the storage means or temporary storage means, and displays this data.

All prior art documents cited in the present specification is incorporated herein by reference.

### BRIEF DESCRIPTION OF THE DRAWINGS

FIG. 1 is a diagram showing the principle of methods for measuring intermolecular interactions using an FAC apparatus. Case I is a procedure for a control substance that does not interact with the immobilized ligands in the column, while Case II is a procedure for a subject of analysis.

FIG. 2 is a diagram summarizing the calculation method for calculating the elution front (V) based on the elution curve obtained in an FAC experiment.

FIG. 3 is a diagram of the composition of a system of the present invention. The detection means is an FAC apparatus or microarray scanner apparatus.

FIG. 4 is a diagram of the composition of a computer in a system of the present invention. Storage Means 6 at least stores Programs 61 to 64 for executing the processes of a system that uses an FAC apparatus and/or Programs 61 to 63 for executing the processes of a system that uses a microarray scanner apparatus. Storage Means (database) 7 stores data on the interaction of a number of sugar chains with various proteins that interact with sugar chains and/or patterns of data combinations of said interaction data.

FIG. 5 is a diagram showing an example of the calculation of the elution front (V) using Program 61-1 of the present invention. (I) in the diagram indicates the calculated elution front.

FIG. 6 is a diagram showing an example in which the V-V₀ values obtained from an FAC experiment are displayed for each sugar chain, then divided into levels in the range of arbitrary thresholds, and encoded.

FIGS. 7-1 to 7-3 are diagrams showing the V-V₀ and Kₐ values for each sugar chain sample as a graph, based on the V-V₀ values obtained from FAC experiments.

FIG. 8 is a diagram showing the sugar chains used for measuring lectin-sugar chain interactions.

FIG. 9 is a diagram showing a six-level evaluation of interactions based on binding strength (V-V₀ value).

FIG. 10 is a diagram showing the encoding of the intensities of lectin-sugar chain interaction using the six-level evaluation.

FIG. 11 is a diagram showing an example of a sugar chain profiling method based on lectin-sugar chain interaction data.

FIG. 12 is a diagram showing the process of reaction of GTMS with a glass surface. The alkoxysilyl groups of GTMS are hydrolyzed by water and become silanol groups. Since these silanol groups are unstable, they partially condense due to changes over time, and form oligomers which subsequently attach to the glass surface via hydrogen bonding. Then, by subjecting the glass to a drying treatment, a dehydration-condensation reaction occurs with the silanol groups on the glass surface, resulting in strong covalent bonding.

FIG. 13 is a diagram showing a substrate, used in the present Examples, on which eight reaction vessels have been formed. The newly designed 8-hole rubber is 1 mm thick, and by adhering it to a slide glass on a specific adjuster, a fluorescence-labeled probe solution can be accurately filled to the surroundings of the spots. The reaction vessels are optimally filled with 50 μL of sample.

FIG. 14 is a conceptual diagram of a lectin array performance experiment in which a Cy3-ASF solution is added to an array onto which two types of lectin have been immobilized.

FIG. 15 is a diagram showing the relationship between the concentration of the lectin solution at the time of immobilization and the fluorescent intensity of the spots. When detecting lectin-sugar chain interactions with a high affinity constant, setting the concentration of the spotted lectin samples to a high concentration of 1 mg/mL or more was revealed to be effective in improving the signal intensity.

FIG. 16 is a diagram showing the detection of lectin-sugar chain interactions and the effect of an inhibiting sugar on the interaction. Strong fluorescence was observed in RCA-120 spots, while moderate fluorescence was observed in EW29(Ch) spots.

FIG. 17 is a diagram showing the effect of an inhibiting sugar on the lectin-sugar chain interaction as a graph. The experiment was carried out in the presence of lactose (a competitively inhibiting sugar). Since the fluorescence intensity of the spots decreases as the concentration of lactose (competitively inhibiting sugar) increases, binding of the fluorescent glycoprotein probe was confirmed to be a sugar-specific binding reaction between the lectin and the sugar chain.

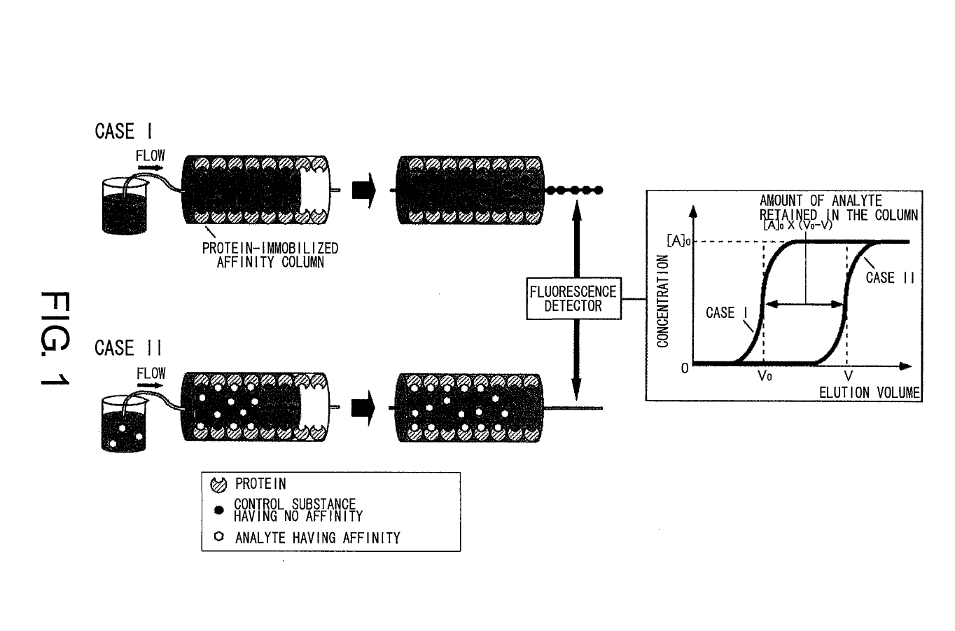

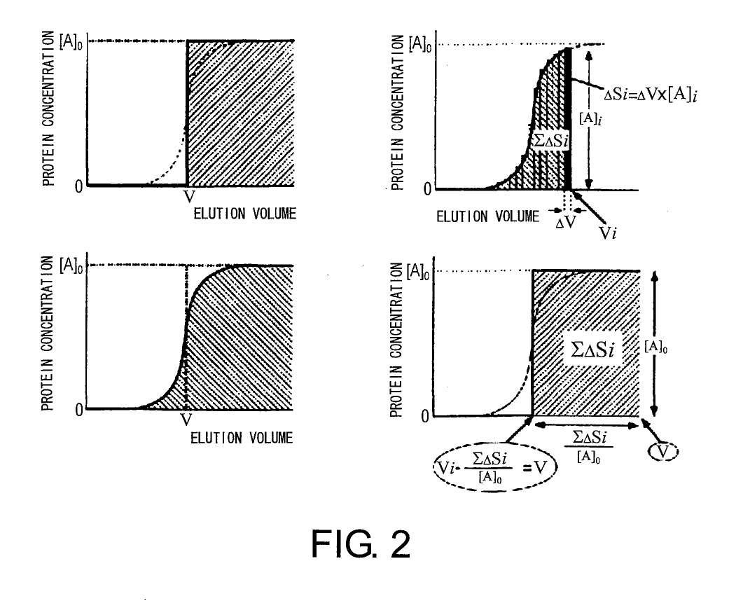

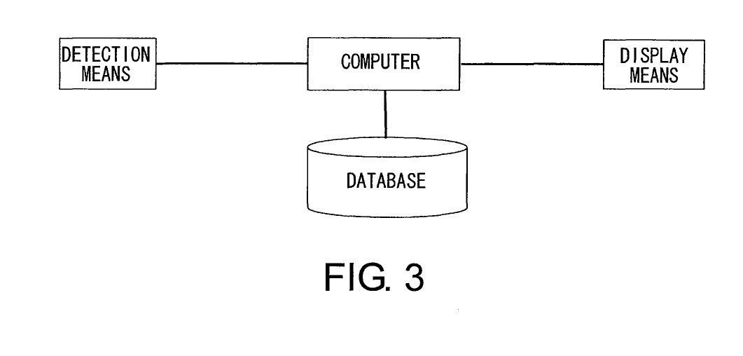

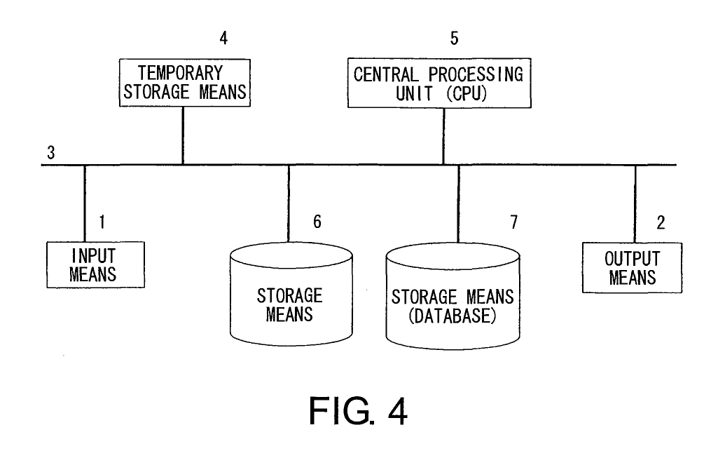

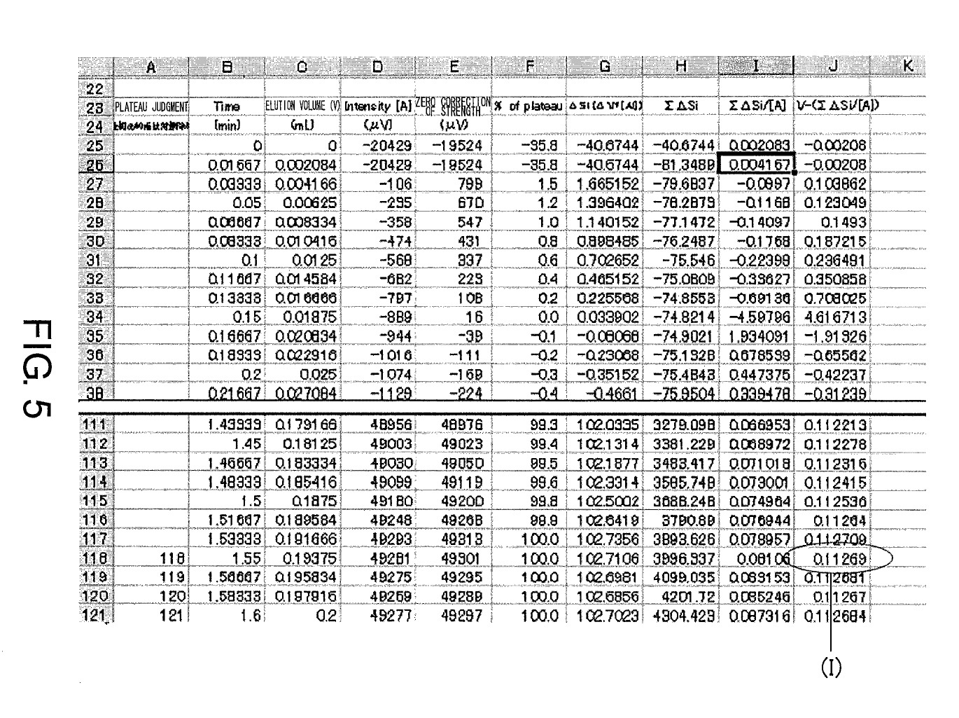

### BEST MODE FOR CARRYING OUT THE INVENTION

Herein below, the present invention will be specifically described with reference to the Examples, but it is not to be construed as being limited thereto.

### [EXAMPLE 1] CONSTRUCTION OF A LECTIN AND SUGAR CHAIN INTERACTION DATABASE USING AN FAC APPARATUS, AND DETERMINATION OF SUGAR CHAIN STRUCTURES

Data on lectin-sugar chain interactions were collected using an automated frontal affinity chromatography apparatus (FAC-1, Shimadzu Corp.) in which two lectin columns were connected in parallel. The lectin columns required for analysis of lectin-sugar chain interactions were prepared according to the method described below:
1. Purified lectins are dissolved in a 0.1 M sodium hydrogen carbonate buffer (pH 8.5).
2. Lectins are immobilized on NHS-activated resins via the primary amino groups in the proteins.
3. Resins are prepared such that the concentration of immobilized lectins is 2 to 9 mg/mL.
4. The lectin-immobilized resins are filled into capsules (inner diameter: 2 mm, length: 10 mm) with a filling volume of 31.4 μL.
5. The capsules are sandwiched between two filters.
6. Two types of capsules, each filled with a lectin-immobilized resin, are protected with a holder and connected to the FAC-1 as lectin columns.

PA sugar chains (pyridylaminated sugar chains), diluted to a concentration sufficiently lower (2.5 nM) than the dissociation constant (K_{d}) of the lectins with the analysis buffer (10 mM Tris-HCl buffer (pH 7.4) containing 0.8% NaCl), were continuously injected, 300 μL at a time, at a flow rate of 0.125 mL/min into the two equilibrated lectin columns. Injection was carried out using an autosampler and each sample was alternately measured for five minutes at a time. Elution of PA sugar chains from columns was detected using a fluorescence detector (RF10AXL, Shimadzu Corp., excitation wavelength/fluorescence wavelength=310 nm/380 nm).

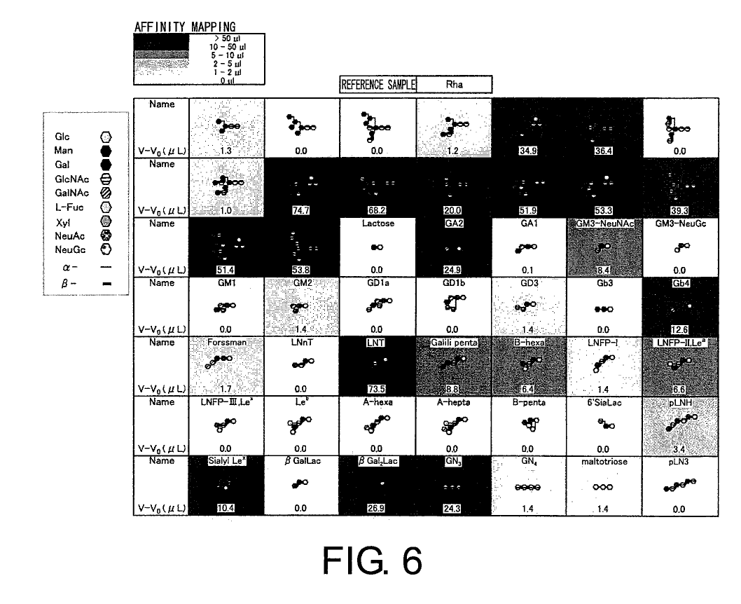

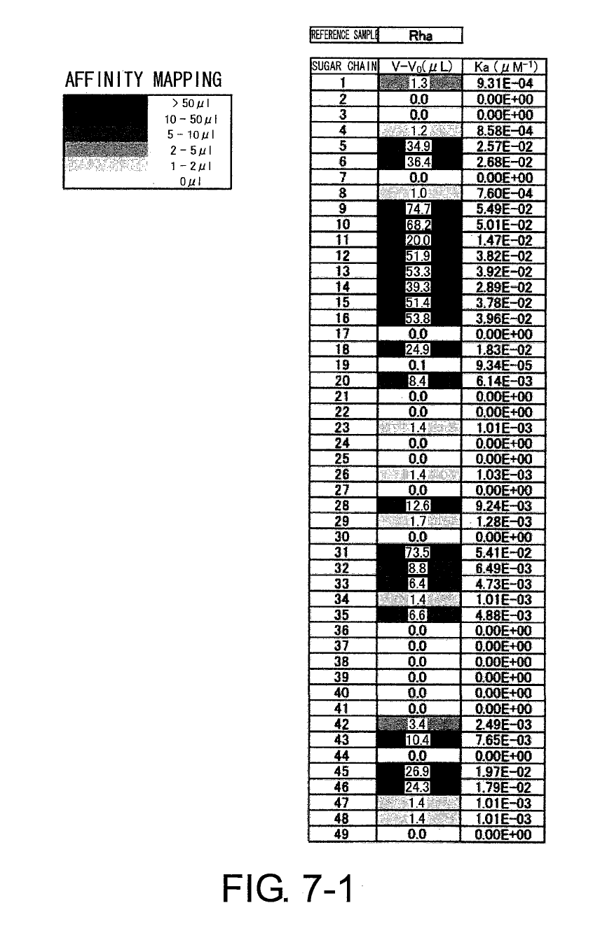

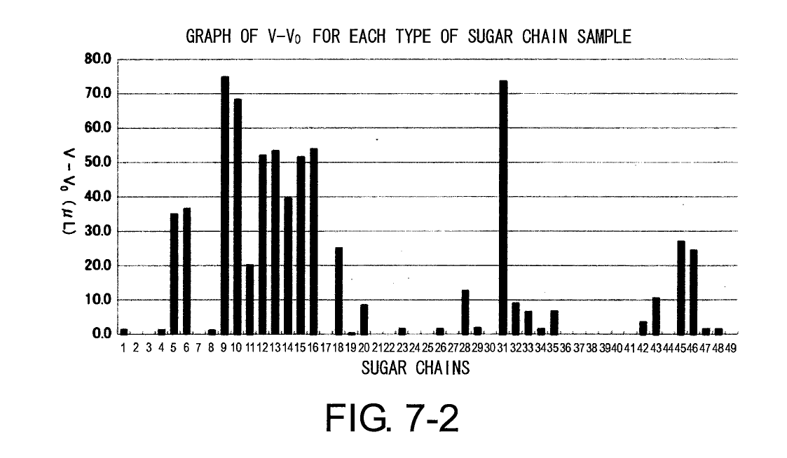

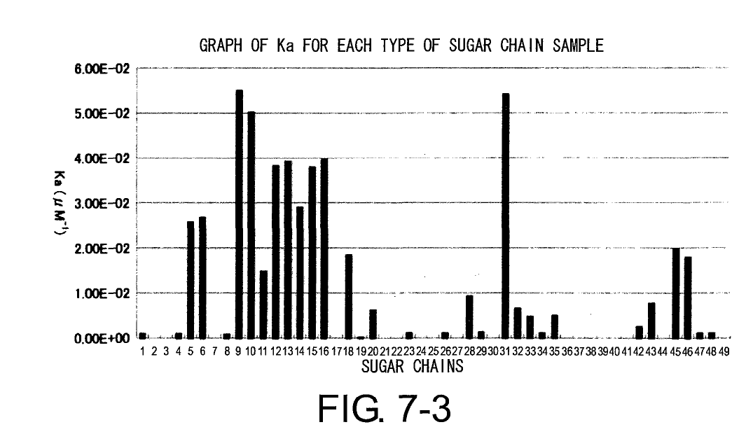

Interaction data was obtained as a delay (V-V₀) in the elution front (V) of interacting sugar chains or as an affinity constant (Kₐ) between sugar chains and lectins when the elution front (V₀) of a sugar chain that does not interact with lectin (PA rhamnose) is taken as the standard. Specifically, data detected using the fluorescence detector were controlled with the general-purpose HPLC control software “LC Solution”, manufactured by Shimadzu Corp (which carries out basic operations such as production of the analysis method, storage of data, and writing from data files to text files). Following completion of a series of experiments, data were written from data files to text files using “LC Solution”, and analytical calculations were carried out using the originally developed Excel-based software, “FAC Analyzer Ver. 3.17” (FIGS. 6 and 7). Use of this software enables simultaneous and automatic calculation of the elution front (V) of each sample (Arata, Y, Hirabayashi, J., and Kasai, K., J. Chromatogr. A905, 335-343, 2001; Arata, Y, Hirabayashi, J. and Kasai, K., J. Biol. Chem. 276, 3068-3077, 2001), batch calculation of the delay (V-V₀ values) in the elution of each sample with respect to a reference sugar chain sample, automatic calculation of Kₐ values, display of a list of chromatograms, display of V-V₀ and Kₐ values corresponding to each sugar chain sample, and encodings of the strength of the interactions of each sugar chain sample. In addition, this software also incorporates a function that separates values into levels in the range of thresholds when arbitrary threshold setting values are entered. Using this function in the present Example, V-V₀ values were separated into levels by taking the following threshold values as standards, and numbers (0 to 5) were assigned (encoded).

| Current Tentative Threshold Values | (V-V₀, μL) |
|---|---|
| 1 or less | Level 0 |
| Less than 2 | Level 1 |
| 2 to less than 5 | Level 2 |
| 5 to less than 10 | Level 3 |
| 10 to less than 50 | Level 4 |
| 50 or more | Level 5 |

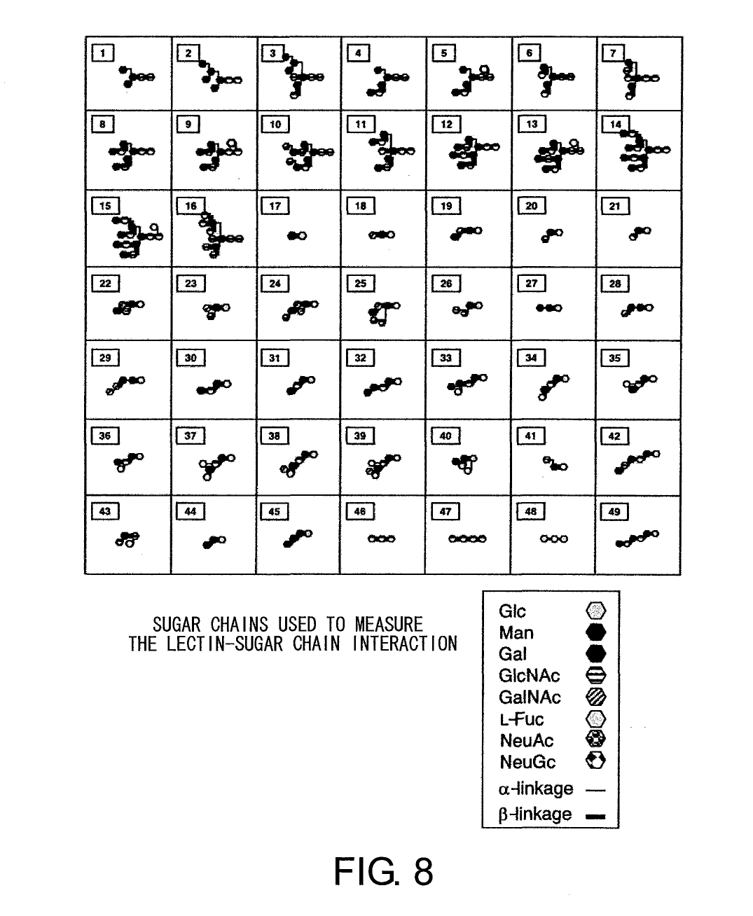

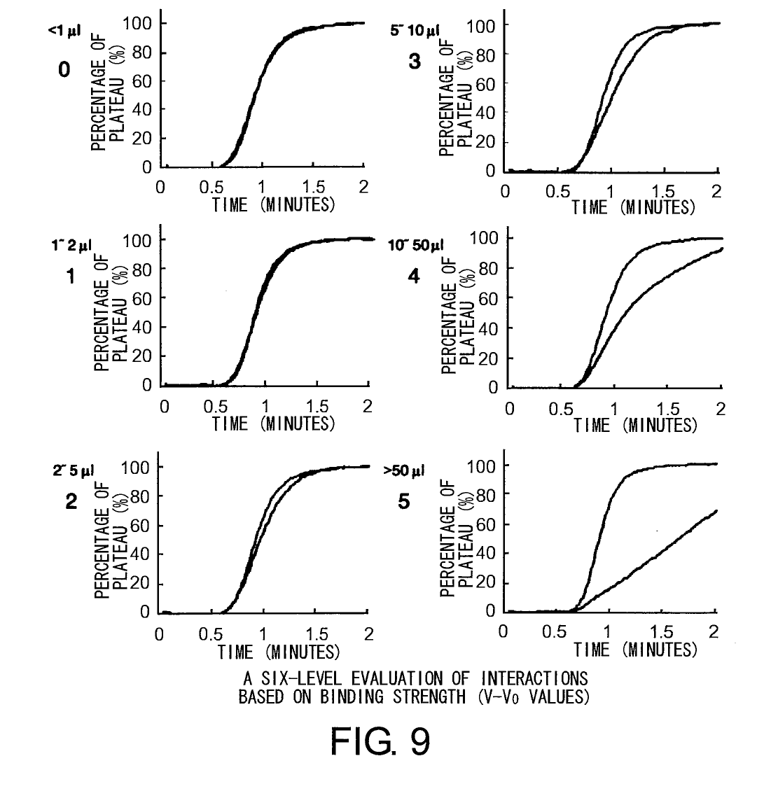

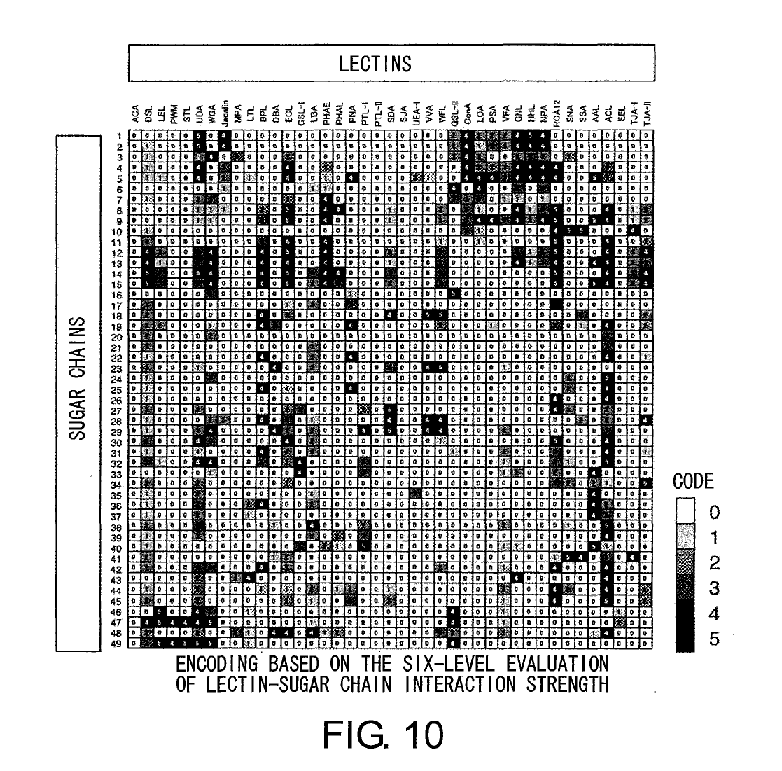

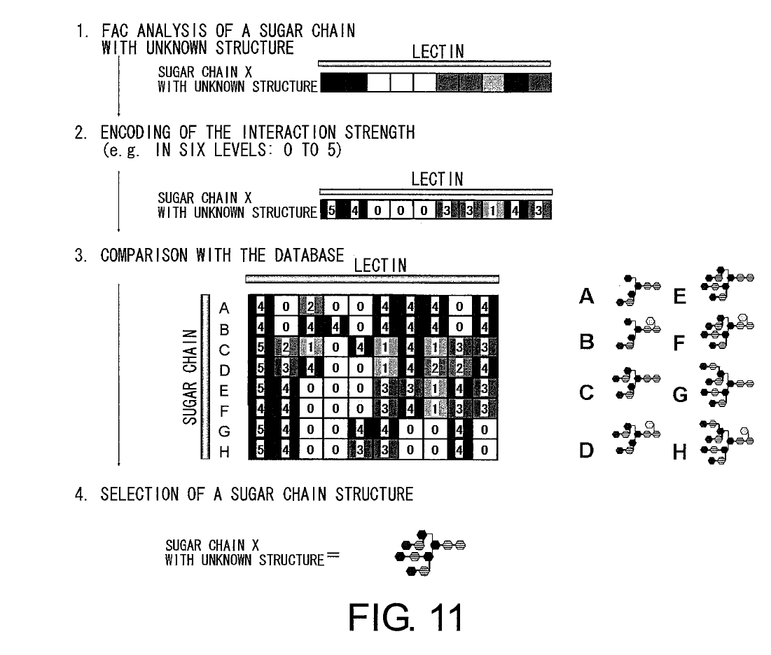

Specifically, the interactions between 41 types of plant and bacterial lectins and 49 types of PA sugar chains (FIG. 8) were measured. As a result, V-V₀, which are quantitative interaction data, could be obtained for 2009 interactions. The interaction data obtained here were evaluated according to a six-level evaluation, based on binding strength (V-V₀ values), and encoded with “0 to 5” (FIGS. 9 and 10) to construct a database. Sugar chain structures could be identified using the procedure shown in FIG. 11. As a result, it was revealed that numerous types of sugar chains can be distinguished using existing databases, even with a limited number of lectins. Theoretically, in the aforementioned process, ten types of lectins with different specificity can be used to distinguish 6¹⁰=60,466,176 types of sugar chains, and most of the sugar chain structures that exist in nature can in fact be distinguished. Data obtained from interactions between numerous types of lectins and libraries of numerous types of sugar chain preparations are stored in this database. Thus, the present inventors judge that by using this database, a considerable number of sugar chain structures can be distinguished.

### [EXAMPLE 2] METHOD FOR ESTIMATING THE STRUCTURE OF SUGAR CHAINS WHOSE STRUCTURE IS UNKNOWN USING THE MANHATTAN METHOD

To use the patterns from combinations of interactions between various sugar chains and lectins in order to estimate the structures of sugar chains of unknown structure, a technique was used that calculates the degree of variance (degree of similarity) from the distance between two samples using a pattern search method. To verify this technique, a blind test was carried out using the interaction pattern of a subject sugar chain (query) on the interaction patterns of sugar chains of known structure (database). Specifically, for a subject sugar chain of unknown structure, the patterns of interaction with eight types of lectins were entered. Then, a sugar chain of known structure with a pattern with a low degree of variance (a pattern with a high degree of similarity) with an interaction pattern of the subject sugar chain was searched from the data stored in a database of interactions of sugar chains of known structure with lectins, and this was displayed as the estimated result.

1. Method

(1) Data Pre-Processing

Negative V-V₀ values were replaced with zero. Next, [1] data stored in the database on the interaction of each lectin were converted to relative values. Specifically, a sugar chain having a V-V₀ value ranging from 10 μL to 20 μL was defined as the reference sugar chain for each lectin, and the relative value of each sugar chain was determined by the following equation:

[Equation 8]
Relative value of sugar chain i=(V-V₀ of sugar chain i/V-V₀ of reference sugar chain)×100 (Equation 8)

When there were a number of sugar chains having a V-V₀ value of 10 μL to 20 μL, the sugar chain having the greatest value was used. The relative values for the strength of interaction with each lectin are shown in Table 1.

TABLE 1
Relative V-V₀ values of sugar Chains of known structure
stored in the database (15 sugar Chains, eight lectins)

| | BPL | LCA | PSA | VFA | GNL | NPA | HHL | JACALIN |
|---|---|---|---|---|---|---|---|---|
| SUGAR | 0.00 | 19.17 | 19.48 | 29.03 | 187.34 | 113.37 | 423.19 | 150.00 |
| CHAIN 1 | | | | | | | | |
| SUGAR | 0.00 | 15.83 | 25.97 | 24.73 | 100.00 | 82.89 | 286.23 | 100.00 |
| CHAIN 2 | | | | | | | | |
| SUGAR | 0.00 | 29.17 | 3.46 | 8.60 | 25.95 | 14.44 | 46.38 | 0.00 |
| CHAIN 3 | | | | | | | | |
| SUGAR | 0.00 | 20.00 | 12.12 | 20.43 | 143.67 | 100.00 | 100.00 | 35.63 |
| CHAIN 4 | | | | | | | | |
| SUGAR | 0.00 | 214.17 | 132.03 | 100.00 | 203.16 | 157.22 | 158.70 | 22.41 |
| CHAIN 5 | | | | | | | | |
| SUGAR | 0.00 | 100.00 | 0.00 | 5.38 | 62.03 | 36.36 | 3.62 | 13.22 |
| CHAIN 6 | | | | | | | | |
| SUGAR | 0.00 | 60.00 | 0.00 | 13.98 | 12.66 | 8.02 | 5.80 | 1.72 |
| CHAIN 7 | | | | | | | | |
| SUGAR | 26.52 | 15.83 | 0.00 | 5.38 | 68.35 | 52.41 | 14.49 | 9.77 |
| CHAIN 8 | | | | | | | | |
| SUGAR | 37.02 | 213.33 | 100.00 | 53.76 | 86.08 | 70.59 | 26.81 | 0.00 |
| CHAIN 9 | | | | | | | | |
| SUGAR | 43.65 | 13.33 | 0.00 | 6.45 | 0.63 | 0.00 | 0.00 | 0.00 |
| CHAIN 10 | | | | | | | | |
| SUGAR | 100.00 | 0.00 | 0.00 | 3.23 | 45.57 | 39.04 | 7.25 | 0.00 |
| CHAIN 11 | | | | | | | | |
| SUGAR | 66.85 | 0.00 | 0.00 | 7.53 | 80.38 | 47.59 | 9.42 | 0.00 |
| CHAIN 12 | | | | | | | | |
| SUGAR | 117.68 | 0.00 | 0.00 | 0.00 | 0.00 | 0.00 | 0.00 | 0.00 |
| CHAIN 13 | | | | | | | | |
| SUGAR | 114.36 | 0.00 | 0.00 | 0.00 | 0.00 | 0.00 | 0.00 | 0.00 |
| CHAIN 14 | | | | | | | | |
| SUGAR | 0.00 | 0.00 | 0.00 | 0.00 | 0.00 | 0.00 | 0.00 | 0.00 |
| CHAIN 15 | | | | | | | | |

[2] Relative values of interaction data between subject sugar chains and lectins were calculated in the same manner by setting V-V₀ of the reference sugar chains defined in (1) for each lectin as the reference value (however, the V-V₀ of the reference sugar chain is not necessarily within 10 μL to 20 μL). Relative values for the strength of interaction with each lectin are shown in Table 2.

TABLE 2
Relative V-V₀ values of subject sugar Chains (13 sugar Chains, eight lectins)

| | | BPL | LCA | PSA | VFA | GNL | NPA | HHL | JACALIN |
|---|---|---|---|---|---|---|---|---|---|
| SUBJECT | SUGAR | 0.00 | 24.23 | 16.22 | 10.40 | 324.00 | 138.18 | 438.01 | 172.83 |
| SUGAR | CHAIN A | | | | | | | | |
| CHAIN | SUGAR | 0.00 | 34.34 | 16.99 | 27.04 | 100.00 | 83.26 | 211.81 | 100.00 |
| | CHAIN B | | | | | | | | |
| | SUGAR | 22.95 | 35.96 | 9.15 | 62.83 | 0.00 | 100.00 | 100.00 | 4.79 |
| | CHAIN C | | | | | | | | |
| | SUGAR | 11.91 | 509.91 | 57.41 | 100.00 | 327.16 | 310.25 | 201.62 | 21.28 |
| | CHAIN D | | | | | | | | |
| | SUGAR | 0.00 | 100.00 | 6.67 | 26.69 | 27.17 | 37.75 | 46.20 | 44.82 |
| | CHAIN E | | | | | | | | |
| | SUGAR | 0.00 | 64.07 | 4.97 | 0.00 | 68.90 | 3.72 | 0.00 | 0.00 |
| | CHAIN F | | | | | | | | |
| | SUGAR | 47.57 | 2.92 | 2.45 | 10.34 | 71.33 | 60.05 | 62.00 | 26.63 |
| | CHAIN G | | | | | | | | |
| | SUGAR | 35.09 | 517.71 | 100.00 | 46.60 | 123.13 | 111.52 | 66.03 | 30.85 |
| | CHAIN H | | | | | | | | |
| | SUGAR | 38.53 | 22.75 | 5.34 | 50.76 | 0.00 | 0.00 | 14.81 | 14.33 |
| | CHAIN I | | | | | | | | |
| | SUGAR | 100.00 | 20.54 | 6.82 | 46.48 | 0.00 | 61.70 | 58.17 | 15.70 |
| | CHAIN J | | | | | | | | |
| | SUGAR | 64.18 | 1.02 | 1.48 | 10.82 | 89.22 | 70.53 | 64.85 | 15.65 |
| | CHAIN K | | | | | | | | |
| | SUGAR | 127.48 | 3.92 | 3.59 | 32.99 | 0.00 | 0.00 | 37.05 | 20.05 |
| | CHAIN L | | | | | | | | |
| | SUGAR | 0.00 | 0.00 | 0.77 | 13.40 | 0.00 | 0.00 | 25.53 | 18.31 |
| | CHAIN M | | | | | | | | |

(2) Method for Searching for Patterns (Method for Calculating the Degree of Variance)

The multivariate distance between two points was determined for the interaction patterns of the subject sugar chains, and all sugar chains of known structure contained in the database and models (groups) with a low degree of variance (models (groups) with a high degree of similarity) were extracted. The Manhattan distance was used as the distance scale to calculate the degree of variance (degree of similarity).

Manhattan Distance:

Multivariate distance d_{ab} between sugar chain a and sugar chain b is calculated from the difference between the m variable of each using the following equation:

$$[ Equation 9 ]$$

$${d}_{ab} = {\sum }_{j}^{m} \vert {x}_{aj} - {x}_{bj} \vert \\ \\ ( a \text{:} subject sugar chain , b \text{:} sugar chain of \\ known structure , j \text{:} lectin , m \text{:} number of \\ lectins ) \qquad ( Equation 9 )$$

2. Results and Discussion

Sugar chains in the database were extracted in order of lowest degree of variance (in order of greatest similarity) and compared with the answers of the blind test. The results of the estimated structures of the subject sugar chains are shown in Table 3.

TABLE 3

| | | SUBJECT SUGAR CHAIN | | | | | | |
|---|---|---|---|---|---|---|---|---|
| | | SUGAR | SUGAR | SUGAR | SUGAR | SUGAR | SUGAR | SUGAR |
| | | CHAIN A | CHAIN B | CHAIN C | CHAIN D | CHAIN E | CHAIN F | CHAIN G |
| CORRECT ANSWER | | SUGAR | SUGAR | SUGAR | SUGAR | SUGAR | SUGAR | SUGAR |
| | | CHAIN 1 | CHAIN 2 | CHAIN 4 | CHAIN 5 | CHAIN 6 | CHAIN 7 | CHAIN 8 |
| ORDER OF | 1 | SUGAR | SUGAR | SUGAR | SUGAR | SUGAR | SUGAR | SUGAR |
| MATCHING | | CHAIN 1 | CHAIN 2 | CHAIN 4 | CHAIN 5 | CHAIN 6 | CHAIN 7 | CHAIN 8 |
| | 2 | SUGAR | SUGAR | SUGAR | SUGAR | SUGAR | SUGAR | SUGAR |
| | | CHAIN 2 | CHAIN 4 | CHAIN 3 | CHAIN 9 | CHAIN 3 | CHAIN 6 | CHAIN 12 |
| | 3 | SUGAR | SUGAR | SUGAR | SUGAR | SUGAR | SUGAR | SUGAR |
| | | CHAIN 4 | CHAIN 1 | CHAIN 8 | CHAIN 4 | CHAIN 7 | CHAIN 15 | CHAIN 11 |
| | 4 | SUGAR | SUGAR | SUGAR | SUGAR | SUGAR | SUGAR | SUGAR |
| | | CHAIN 5 | CHAIN 8 | CHAIN 7 | CHAIN 2 | CHAIN 8 | CHAIN 3 | CHAIN 3 |
| | 5 | SUGAR | SUGAR | SUGAR | SUGAR | SUGAR | SUGAR | SUGAR |
| | | CHAIN 8 | CHAIN 3 | CHAIN 10 | CHAIN 1 | CHAIN 15 | CHAIN 8 | CHAIN 10 |
| | 6 | SUGAR | SUGAR | SUGAR | SUGAR | SUGAR | SUGAR | SUGAR |
| | | CHAIN 3 | CHAIN 6 | CHAIN 15 | CHAIN 6 | CHAIN 10 | CHAIN 10 | CHAIN 4 |
| | 7 | SUGAR | SUGAR | SUGAR | SUGAR | SUGAR | SUGAR | SUGAR |
| | | CHAIN 12 | CHAIN 12 | CHAIN 12 | CHAIN 8 | CHAIN 4 | CHAIN 12 | CHAIN 6 |
| | 8 | SUGAR | SUGAR | SUGAR | SUGAR | SUGAR | SUGAR | SUGAR |
| | | CHAIN 8 | CHAIN 7 | CHAIN 6 | CHAIN 3 | CHAIN 11 | CHAIN 11 | CHAIN 15 |
| | 9 | SUGAR | SUGAR | SUGAR | SUGAR | SUGAR | SUGAR | SUGAR |
| | | CHAIN 7 | CHAIN 15 | CHAIN 11 | CHAIN 7 | CHAIN 12 | CHAIN 14 | CHAIN 14 |
| | 10 | SUGAR | SUGAR | SUGAR | SUGAR | SUGAR | SUGAR | SUGAR |
| | | CHAIN 15 | CHAIN 11 | CHAIN 14 | CHAIN 12 | CHAIN 14 | CHAIN 13 | CHAIN 7 |
| | 11 | SUGAR | SUGAR | SUGAR | SUGAR | SUGAR | SUGAR | SUGAR |
| | | CHAIN 11 | CHAIN 10 | CHAIN 13 | CHAIN 11 | CHAIN 13 | CHAIN 4 | CHAIN 13 |
| | 12 | SUGAR | SUGAR | SUGAR | SUGAR | SUGAR | SUGAR | SUGAR |
| | | CHAIN 10 | CHAIN 9 | CHAIN 9 | CHAIN 10 | CHAIN 9 | CHAIN 9 | CHAIN 2 |
| | 13 | SUGAR | SUGAR | SUGAR | SUGAR | SUGAR | SUGAR | SUGAR |
| | | CHAIN 14 | CHAIN 5 | CHAIN 2 | CHAIN 15 | CHAIN 2 | CHAIN 2 | CHAIN 9 |
| | 14 | SUGAR | SUGAR | SUGAR | SUGAR | SUGAR | SUGAR | SUGAR |
| | | CHAIN 13 | CHAIN 14 | CHAIN 5 | CHAIN 14 | CHAIN 5 | CHAIN 5 | CHAIN 1 |
| | 15 | SUGAR | SUGAR | SUGAR | SUGAR | SUGAR | SUGAR | SUGAR |
| | | CHAIN 9 | CHAIN 13 | CHAIN 1 | CHAIN 13 | CHAIN 1 | CHAIN 1 | CHAIN 5 |

SUBJECT SUGAR CHAIN

| | | SUGAR | SUGAR | SUGAR | SUGAR | SUGAR | SUGAR |
|---|---|---|---|---|---|---|---|
| | | CHAIN H | CHAIN I | CHAIN J | CHAIN K | CHAIN L | CHAIN M |
| CORRECT ANSWER | | SUGAR | SUGAR | SUGAR | SUGAR | SUGAR | SUGAR |
| | | CHAIN 9 | CHAIN 10 | CHAIN 11 | CHAIN 12 | CHAIN 13 | CHAIN 15 |
| ORDER OF | 1 | SUGAR | SUGAR | SUGAR | SUGAR | SUGAR | SUGAR |
| MATCHING | | CHAIN 9 | CHAIN 10 | CHAIN 11 | CHAIN 12 | CHAIN 13 | CHAIN 15 |
| | 2 | SUGAR | SUGAR | SUGAR | SUGAR | SUGAR | SUGAR |
| | | CHAIN 5 | CHAIN 15 | CHAIN 14 | CHAIN 8 | CHAIN 14 | CHAIN 10 |
| | 3 | SUGAR | SUGAR | SUGAR | SUGAR | SUGAR | SUGAR |
| | | CHAIN 4 | CHAIN 7 | CHAIN 13 | CHAIN 11 | CHAIN 10 | CHAIN 3 |
| | 4 | SUGAR | SUGAR | SUGAR | SUGAR | SUGAR | SUGAR |
| | | CHAIN 6 | CHAIN 3 | CHAIN 10 | CHAIN 4 | CHAIN 11 | CHAIN 7 |
| | 5 | SUGAR | SUGAR | SUGAR | SUGAR | SUGAR | SUGAR |
| | | CHAIN 8 | CHAIN 14 | CHAIN 3 | CHAIN 3 | CHAIN 15 | CHAIN 14 |
| | 6 | SUGAR | SUGAR | SUGAR | SUGAR | SUGAR | SUGAR |
| | | CHAIN 12 | CHAIN 13 | CHAIN 8 | CHAIN 10 | CHAIN 3 | CHAIN 13 |
| | 7 | SUGAR | SUGAR | SUGAR | SUGAR | SUGAR | SUGAR |
| | | CHAIN 3 | CHAIN 8 | CHAIN 12 | CHAIN 6 | CHAIN 12 | CHAIN 8 |
| | 8 | SUGAR | SUGAR | SUGAR | SUGAR | SUGAR | SUGAR |
| | | CHAIN 7 | CHAIN 11 | CHAIN 15 | CHAIN 14 | CHAIN 7 | CHAIN 11 |
| | 9 | SUGAR | SUGAR | SUGAR | SUGAR | SUGAR | SUGAR |
| | | CHAIN 11 | CHAIN 12 | CHAIN 7 | CHAIN 13 | CHAIN 8 | CHAIN 6 |
| | 10 | SUGAR | SUGAR | SUGAR | SUGAR | SUGAR | SUGAR |
| | | CHAIN 2 | CHAIN 6 | CHAIN 6 | CHAIN 15 | CHAIN 6 | CHAIN 12 |
| | 11 | SUGAR | SUGAR | SUGAR | SUGAR | SUGAR | SUGAR |
| | | CHAIN 10 | CHAIN 4 | CHAIN 4 | CHAIN 7 | CHAIN 4 | CHAIN 4 |
| | 12 | SUGAR | SUGAR | SUGAR | SUGAR | SUGAR | SUGAR |
| | | CHAIN 15 | CHAIN 9 | CHAIN 9 | CHAIN 9 | CHAIN 9 | CHAIN 9 |
| | 13 | SUGAR | SUGAR | SUGAR | SUGAR | SUGAR | SUGAR |
| | | CHAIN 14 | CHAIN 2 | CHAIN 2 | CHAIN 2 | CHAIN 2 | CHAIN 2 |
| | 14 | SUGAR | SUGAR | SUGAR | SUGAR | SUGAR | SUGAR |
| | | CHAIN 13 | CHAIN 5 | CHAIN 1 | CHAIN 1 | CHAIN 1 | CHAIN 1 |
| | 15 | SUGAR | SUGAR | SUGAR | SUGAR | SUGAR | SUGAR |
| | | CHAIN 1 | CHAIN 1 | CHAIN 5 | CHAIN 5 | CHAIN 5 | CHAIN 5 |

The results of the experiment revealed that it is possible to very accurately estimate sugar chain structures from affinity patterns in a database storing data on the interactions of sugar chains of known structure, and affinity patterns of sugar chains of unknown structure.

### [EXAMPLE 3] ANALYSIS OF INTERACTIONS BETWEEN SUGAR CHAINS AND LECTINS USING A LECTIN ARRAY

(1) Preparation of Fluorescence-Labeled Glycoprotein Probe (Cy3-ASF)

Fluorescence-labeled glycoprotein probes were prepared by fluorescently labeling asialofetuin (Sigma, hereinbelow ASF) using Cy3 Mono-reactive Dye (Amersham-Pharmacia, hereinbelow Cy3), which is a fluorescent dye with a maximum absorption wavelength of around 550 nm. ASF is known to have three N-linked sugar chains and three O-linked sugar chains per molecule, and a sugar chain structure in which the sialic acid cap of the non-reducing terminal in the sugar chains is partially removed. After preparing ASF in a 0.1 M carbonate buffer (pH 9.3) such that the final concentration is 1 mg/mL, 1 mL was mixed with 1.0 mg of Cy3 powder and allowed to react in the dark for one hour while stirring occasionally.

Next, free Cy3 and Cy3-ASF were separated and recovered by gel filtration chromatography using Sephadex G-25 as the carrier and the concentration and fluorescence labeling efficiency were measured for the purified Cy3-ASF using a spectrophotometer. Yield based on proteins was 35% to 40% and the fluorescence labeling efficiency (number of fluorescent dyes per protein molecule) was approximately 3.0.

(2) Coating of GTMS onto Slide Glasses

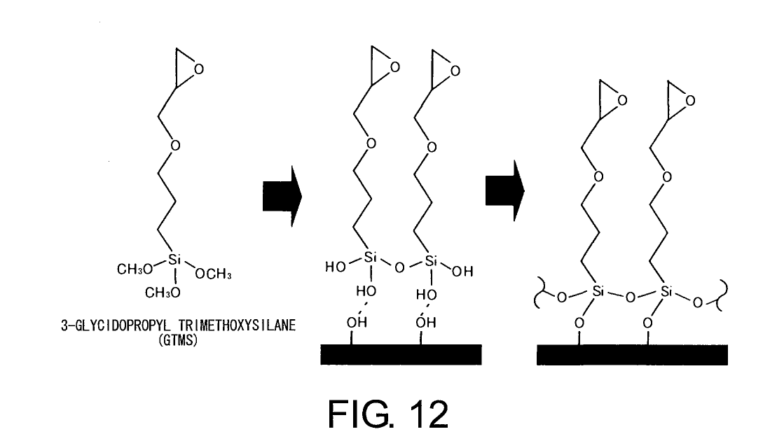

Lectins were immobilized onto the glass surface using slide glasses coated with 3-glycidoxypropyl trimethoxysilane (Shin-Etsu Silicone, hereinbelow GTMS) (FIG. 12) which comprises an epoxy group as the active group. GTMS coating was carried out using slide glasses manufactured by Matsunami Glass Industry Ltd, according to the following procedure: The slide glasses were immersed in a 10% KOH/MeOH solution and allowed to stand for one hour while shaking the container to treat the glass surface. After washing with a sufficient amount of purified water (MilliQ water), they were dried in an oven at 60° C. Next, the slide glasses were immersed in a 2% GTMS acetone solution and reacted in the dark for one hour while shaking the container. After the reaction, they were dried for eight hours in an oven at 110° C., washed with a sufficient amount of purified water, and dried.

(3) Immobilization of Lectins onto Slide Glasses

Lectins were spotted onto the GTMS-coated slide glasses of (2). STAMPMAN, manufactured by Nippon Laser Electronics Ltd., was used as the microarray spotter and spots with a diameter of approximately 0.6 mm to 0.7 mm were arranged onto the slide glasses by using a stamping pin with a tip diameter of 0.40 mm. Each spotted lectin was dissolved in a pH 7.4 phosphate-buffered saline (hereinbelow PBS) such that the concentration was 1 mg/mL (partially 0.5 mg/mL depending on the lectin). These solutions were placed in each well of a 96-well PCR microtiter plate (Corning) in 10 μL aliquots and plates were placed on the microarray spotter.

During the process of immobilizing lectins onto the slide glasses, the following conditions were stored in the memory of the computer attached to the microarray spotter to execute a stamping pin operating program: First, the stamping pin was immersed for one second in the immobilization sample solution contained in the 96-well PCR microtiter plate. It was then lifted out and contacted for one second to a predetermined location on the slide glass surface. After repeating this operation for each spot, and spotting four spots from the same sample solution in a horizontal row, the stamping pin was washed. During the washing step, the tip of the stamping pin was immersed for two seconds in a 0.05% SDS solution, the stamping pin was then dried for 15 seconds in a vacuum apparatus, then after immersion for two seconds in purified water, it was dried for 15 seconds in the vacuum apparatus. After a final immersion for two seconds in ethanol, it was dried for 15 seconds in the vacuum apparatus.

In this Example, a total of five types of proteins were spotted, consisting of four types of lectins with various sugar-binding specificities (Ricinus communis agglutinin (hereinbelow RCA-120), Sambucus sieboldiana agglutinin (hereinbelow SSA), xylane-binding domain of xylanase derived from recombinant actinomycetes (hereinbelow XBD), and C-terminal domain derived from recombinant earthworm 29 kDa lectin (hereinbelow EW29 (Ch)) and one type of negative control (bovine serum albumin (hereinbelow BSA)). RCA-120 and BSA were purchased from Sigma, SSA was purchased from Seikagaku Corp., and the XBD and EW29 (Ch) used were expressed and purified from E. coli in the laboratory of the present inventors.

(4) Blocking of Non-Spotted Surfaces

After immobilizing the lectin solutions onto the glass surfaces, which were reacted for one hour after spot treatment, the unbound lectins were washed. Washing was carried out by pipetting, as though spraying, a PBS solution comprising 0.1% Tween20 (PBST) several times onto the slide glasses, followed by further sufficient washing using PBS.

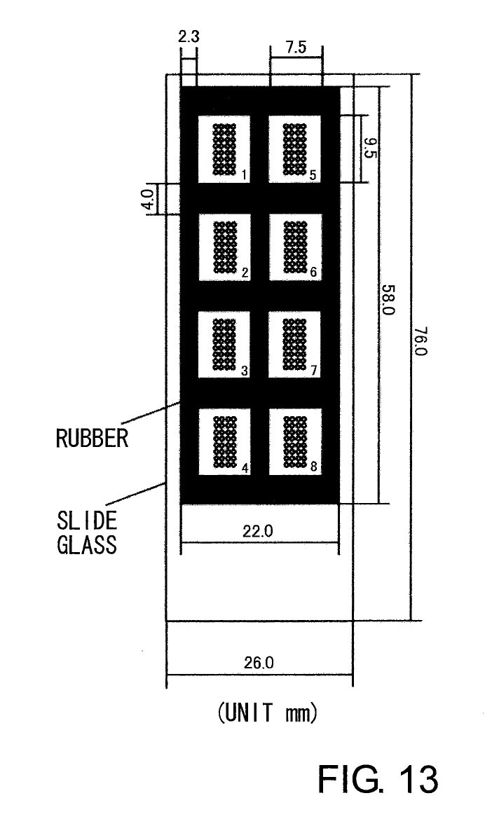

An 8-hole rubber designed and developed by the present inventors was affixed to a predetermined location on the slide glass after lectin immobilization to prepare eight reaction vessels (FIG. 13). This 8-hole rubber is made of a black silicon rubber with a thickness of 1 mm with eight 9.5×7.5 mm rectangular holes are formed in an orderly arrangement therein. When affixed to the slide glasses, the 8-hole rubber can form eight reaction vessels. Adding about 50 μL of sample to a reaction vessel can sufficiently fill the inside with sample solution.

Since epoxy groups, which are active groups, are present on the glass surface in areas other than where lectins were spotted, a blocking procedure was carried out on the non-spotted surfaces. High-purity BSA (Sigma) was used as the blocking agent. Blocking of the non-spotted surfaces on the slide glass was carried out by filling the eight reaction vessels with 50 μL each of a PBS solution comprising 1% BSA and allowing the vessels to stand for one hour at 4° C. in a storage container with humidity maintained at 90% or more. Care was taken to prevent the glass surface from drying out during the reaction.

Next, the blocking solution was removed from the slide glasses, the glass surfaces were sufficiently washed using PBS, and the moisture was eliminated. To prevent the protein denaturation caused by the drying out of the glass surface and the increase of the background that accompanies drying, the experiment was moved on to the next procedure as soon as possible after protein immobilization.

(5) Addition of the Probe Solution and Scanning

A fluorescence-labeled glycoprotein probe solution, the interaction analysis of which is desired, was added to the reaction vessels on the lectin-immobilized slide glasses prepared in (4). The fluorescence-labeled glycoprotein probes were prepared by dissolution in PBS such that the final concentration was 10 μg/mL, and 50 μL was dropped into each reaction vessel.

The reaction vessels were left to stand until the lectin-sugar chain reaction had reached equilibrium, then an excitation light was injected from the edge of the slide glasses using a GTMAS Scan III (Nippon Laser Electronics), which is an evanescent excitation-type microarray scanner, and the emitted fluorescent light generated by excitation was detected using an ICCD (charge coupled device with image intensifier) camera positioned on the lower surface of the slide glasses. Fluorescent images corresponding to nearly the entire surface of the slide glasses were scanned, and the obtained images were saved as TIFF files (approximately 100 megabytes per image). The parameters during scanning were standardized as a gain of “5000 times”, number of integration of “four times” and an exposure time of “33 msec”.

(6) Digitization of the Scanned Images

Array-Pro Analyzer (Version 4.0 for Windows (registered trademark), Media Cybernetics), which is a commercially available analysis software for microarrays, was used to digitize the scanned images. The brightness of each spot was calculated using the aforementioned analysis software, and the brightness of the non-spotted areas was used as a background value. The difference obtained by subtracting the background value from the brightness of each spot was defined as the net brightness value, and mean values and standard deviations were calculated for each horizontal row of four spots derived from the same sample.

Subsequently, probe binding to each lectin sample was evaluated using this mean brightness value of the four spots derived from the same sample. The performance of each lectin array shown below was evaluated after going through the series of operations (2) to (6).

(7) Evaluation of the Performance of GTMS-Coated Slide Glasses

The performance of the GTMS-coated slide glasses, prepared as described above, was evaluated by comparison with existing slide glasses (six types). Specifically, Cy3-prelabeled lectins (100 μg/mL) were immobilized in the form of an array onto each surface-coated slide glass, and after having gone through steps (3) to (6), the S/N ratios were calculated from the brightness value of the spotted areas (S) and the brightness value of the non-spotted areas (N). As a result, as shown in Table 4, although the brightness value of the GTMS-coated slide glasses prepared in step (2) remained at around one-half that of slide glass A, which showed the highest brightness value, since the background is extremely low, its S/N was 16.1 and showed the best value from among the slide glasses evaluated this time.

TABLE 4
PERFORMANCE EVALUATION OF EACH SLIDE GLASS
100 μg/ml Cy3 RCA-120 in 30% glycerol/PBS

| | MEAN VALUE OF 4 SPOTS | MEAN VALUE OF 4 BLANKS | |
|---|---|---|---|
| | (GAIN × 1000)* | (GAIN × 1000) | S/N RATIO |
| COMMERCIALLY AVAILABLE SLIDE GLASS A | 60617 | 5971 | 10.2 |
| COMMERCIALLY AVAILABLE SLIDE GLASS B | 52059 | 4013 | 13.0 |
| COMMERCIALLY AVAILABLE SLIDE GLASS C | 36462 | 2865 | 12.7 |
| GTMS SLIDE GLASS | 28220 | 1753 | 16.1 |
| COMMERCIALLY AVAILABLE SLIDE GLASS D | 13838 | 4520 | 3.1 |
| COMMERCIALLY AVAILABLE SLIDE GLASS E | 12802 | 3105 | 4.1 |
| COMMERCIALLY AVAILABLE SLIDE GLASS F | 5902 | 1621 | 3.6 |

*COMPARISON OF THE MEAN BRIGHTNESS VALUES OF THE SAME Cy3-LABELED LECTIN SPOTS

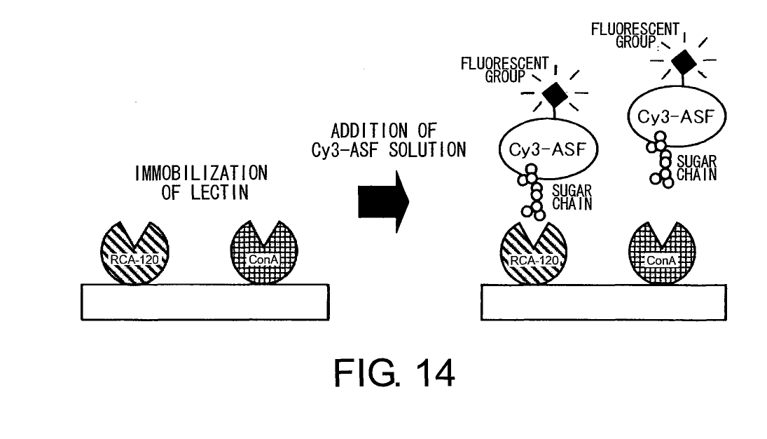

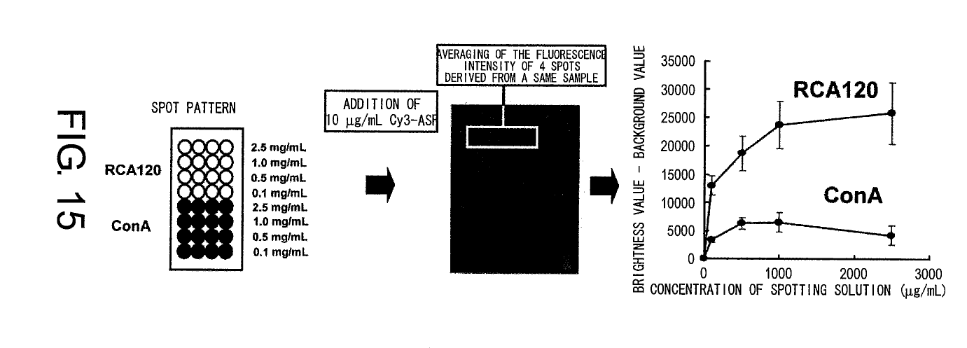

(8) Study of the Concentrations of Immobilized Lectins on the Arrays (FIGS. 14 and 15)

RCA-120 and ConA are typical lectins known to have high affinity for complex sugar chains and high-mannose sugar chains, respectively. These lectins were prepared at various concentrations and spotted in the form of an array, with four spots of the same sample arranged horizontally. 50 μL of 10 μg/mL Cy3-ASF were dropped into each of these arrays, and fluorescence was observed with a scanner.

As previously described, ASF is known to have three N-linked sugar chains and three O-linked sugar chains per molecule, and a sugar chain structure in which the sialic acid cap of the non-reducing terminal in the sugar chains is removed, resulting in a protruding lactosamine structure. Therefore, in an experimental system in which Cy3-ASF was added to lectin arrays onto which RCA-120 and ConA were immobilized, it was predicted that RCA-120 would show an extremely strong affinity, while ConA would show a weak affinity.

The experiment results suggest that the RCA-120 spots emitted an intense fluorescence, while the ConA spots only showed a fluorescence intensity of about one-third that of the RCA-120 spots under the same conditions. ConA was thought to bind, albeit weakly, to ASF, which has complex sugar chains, because it can bind to the biantennary N-linked sugar chains, which are considered present in small amounts, even though it cannot bind to the triantennary sugar chains that are mainly present in ASF. In addition, this data also showed that the standard deviation (SD) for four spots derived from the same sample is approximately ±20% (FIG. 15).

Next, representing the relationship between lectin concentration at the time of spotting and fluorescence intensity as a graph revealed a positive correlation between the two, revealing that signal intensity can be effectively improved by increasing the concentration of the lectin sample to be spotted to 1 mg/mL or more. Specifically, the results revealed that interactions between lectins and sugar chains with a small affinity constant (weak binding) can be detected by increasing the concentration of the immobilized lectin (FIG. 15).

(9) Evaluation of the Performance of the Lectin Array

A total of five types of proteins consisting of four types of lectins having various sugar specificities (RCA-120, SSA, XBD, and EW29 (Ch)) and one type of negative control (BSA) were spotted in the form of an array, with four spots arranged horizontally for the same sample. 50 μl of 10 μg/mL Cy3-ASF was dropped to each of these arrays and the fluorescence was observed with a scanner.

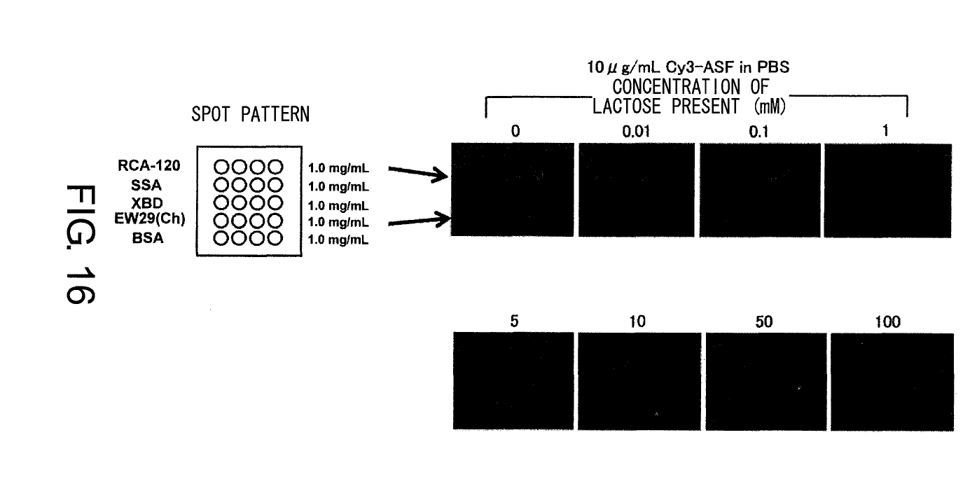

As a result of this experiment, fluorescent signals were observed for the spots of two types of lectins, RCA-120 and EW29 (Ch), which were confirmed by FAC to have an affinity for the lactosamine structure (FIG. 16). In addition, when the fluorescence intensities of each were compared, a strong fluorescence was observed for RCA-120 spots while an intermediate fluorescence was observed for EW29 (Ch) spots, matching the FAC analysis data.

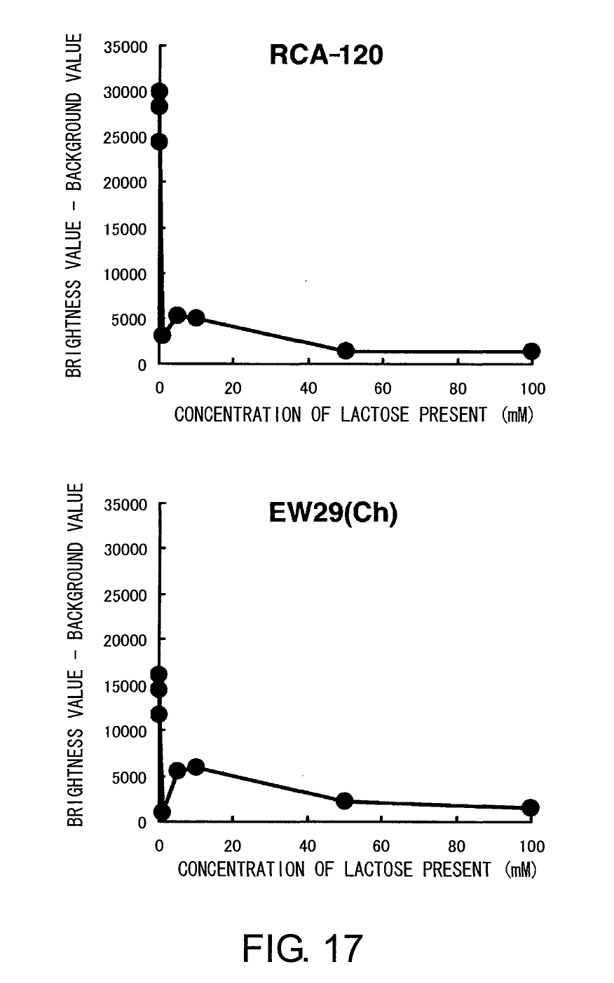

In addition, when a similar experiment was conducted on an array under the same conditions in the presence of lactose (a competitively inhibiting sugar), the fluorescence intensity of the spots was observed to decrease as the concentration of inhibiting sugar increased (FIG. 17). From the above, the binding to fluorescent glycoprotein probes was confirmed to be due to a sugar-specific binding reaction between lectins and sugar chains.

### INDUSTRIAL APPLICABILITY

In past technologies, the existence of a sugar chain structure was estimated from the presence or absence of binding when sugar chains were reacted with anti-sugar-chain antibodies immobilized onto a membrane. However, it is difficult to prepare antibodies against sugar chains that are highly common among organisms, or sugar chains that are present in extremely small amounts in the body. In addition, in reality it is extremely difficult to prepare a large number of antibody libraries against sugar chains, which are known to have an extremely wide diversity. Moreover, in practice it was difficult to determine how to evaluate situations where, in the process of gradually digesting sugar chains enzymatically and reacting them with the membrane on which antibodies were immobilized, the enzymatic digestion did not proceed completely. There is also a shortcoming in that further analyses cannot be performed if an enzyme that cleaves a desired bond cannot be obtained.

Although there have been reports that sugar chain structures can be estimated using five types of lectin, these reports do not use quantitative affinity data, and thus the data that can be obtained using a number of lectins in this range merely indicates a portion of the characteristics within a sugar chain structure, and is incomplete. Moreover, when gradually enzyme-digesting labeled sugar chains comprising only five constituent sugars, there have been reports that sugar chain structures can be estimated by observing changes in the patterns of interaction of five types of lectin. However, when the number of monosaccharides constituting the sugar chain increases, this is considered to be clearly disadvantageous in terms of time, labor, the number of enzymes to be prepared, and such.

In contrast, in the analyses of interactions using the FAC apparatus of the present invention, the structures are estimated using the quantitative information of control interaction data obtained in advance from numerous types of sugar chain standard libraries and numerous types of lectins, and thus sugar chain structures can be estimated or identified with higher accuracy. Analysis using an FAC apparatus does not require enzymatic digestion of labeled sugar chains nor time and effort spent on incubation or blocking. On the other hand, when analyzing interactions using a microarray slide and a microarray scanner, spots can be set at a high density and simultaneous parallel processing of a number of probe solutions is possible, and thus analysis throughput can be increased considerably. In addition, since washing and removing procedures for the probe solution are not performed, the procedures can save on labor and time.

The present invention is expected to accumulate the interaction data forming its basis, select an optimum rationale for sugar chain profilers, create a rationale (prototype) of a sugar chain profiler, develop joint technology with numerous other principles (MS, bio-IT, etc), and such. Moreover, the development and market introduction of a high throughput apparatus that can analyze the profile of sugar chains by time units using extremely small amounts of patient tissue or blood, enabling the immediate and precise diagnosis of diseases and such; the practical application of a sugar chain profiling system capable of substantially specifying and describing sugar chain structures; and the elucidation of life phenomena brought as a result of the popularization thereof are expected from the present invention.

1. A method for analyzing a sugar chain structure, wherein the method comprises the steps of:
(a) introducing a fluorescence-labeled subject sugar chain to an FAC apparatus having parallel columns onto which each of a variety of proteins that interact with a sugar chain are immobilized; and
(b) measuring the interaction of the subject sugar chain with each of the proteins that interact with the sugar chain; wherein
when a combined pattern of a measured interaction of the subject sugar chain with each of the proteins that interact with the sugar chain matches a combined pattern of an interaction of a specific sugar chain with each of the proteins that interact with the sugar chain, taken from control data which comprise the interactions of a number of sugar chains with each of the proteins that interact with the sugar chain, the subject sugar chain is judged to have the same structure as the specific sugar chain.

2. The method of claim 1, wherein a protein that interacts with the sugar chain is a lectin, an enzymatic protein comprising a sugar-binding domain, a cytokine having affinity for a sugar chain, or an antibody that interacts with a sugar chain.

3. A system for analyzing a sugar chain structure using a computer and comprising:
(a) a storage means which stores data on the interaction of a number of sugar chains with a variety of proteins that interact with a sugar chain;
(b) a detection means which, when a fluorescence-labeled subject sugar chain is introduced into an FAC apparatus having parallel columns onto which each of the various proteins that interact with sugar chains is immobilized, detects the fluorescence intensity over time of a label attached to a subject sugar chain eluted from each column;
(c) a means for calculating data on the interaction of a subject sugar chain with each of the proteins that interact with sugar chains, based on an entered fluorescence intensity data, comparing a data combination of said interaction data with a data combination stored in (a), and selecting one or a number of sugar chains of known structure having a matching data combination pattern; and,
(d) a display means for displaying the selection results.

4. The system of claim 3, wherein the arithmetic processing means of step (c) comprises the following (i) or (ii):
(i) a means for calculating the elution volume of a subject sugar chain from each column based on an entered fluorescence intensity data, calculating a difference between said elution volume and a control elution volume, comparing a data combination of said difference with a data combination stored in (a), and selecting one or a number of sugar chains of known structure having a matching data combination pattern; or,
(ii) a means for calculating the elution volume of a subject sugar chain from each column based on an entered fluorescence intensity data, calculating a difference between said elution volume and a control elution volume, calculating an affinity constant for the subject sugar chain with each of the proteins that interact with sugar chains based on said difference, comparing a data combination of said affinity constant with data combination stored in (a), and selecting one or a number of sugar chains of known structure having a matching data combination pattern.

5. The system of claim 3, wherein a protein that interacts with a sugar chain is a lectin, an enzymatic protein having a sugar-binding domain, a cytokine having an affinity for a sugar chain, or an antibody that interacts with a sugar chain.

6. A method for analyzing a sugar chain structure, wherein the method comprises:
(a) a step of contacting a fluorescence-labeled subject sugar chain with a substrate onto which each of a variety of proteins that interact with a sugar chain is immobilized; and
(b) a step of measuring the interaction of the subject sugar chain with each of the proteins that interact with the sugar chain by allowing an excitation light to act without carrying out a washing operation; wherein
when a combined pattern of a measured interaction of the subject sugar chain with each of the proteins that interact with the sugar chain matches a combined pattern of an interaction of a specific sugar chain with each of the proteins that interact with the sugar chain, taken from control data which comprise the interactions of a number of sugar chains with each of the proteins that interact with the sugar chain, the subject sugar chain is judged to have the same structure as the specific sugar chain.

7. The method of claim 6, wherein the excitation light is an evanescent wave.

8. The method of claim 6, wherein a protein that interacts with a sugar chain is a lectin, an enzymatic protein having a sugar-binding domain, a cytokine having an affinity for a sugar chain, or an antibody that interacts with a sugar chain.

9. A system for analyzing a sugar chain structure using a computer and comprising:
(a) a storage means which stores data on the interaction of a number of sugar chains with a variety of proteins that interact with a sugar chain;
(b) a detection means which, when a fluorescence-labeled subject sugar chain is contacted with a substrate onto which each of the various proteins that interact with sugar chains are immobilized, detects the intensity of an excited fluorescence after an incident excitation light has been shone on the substrate, without carrying out a washing procedure;
(c) a means for taking a data combination of the detected fluorescence intensity, comparing it with data stored in (a), and selecting one or a number of sugar chains of known structure having a matching data combination pattern; and
(d) a display means for displaying the selection results.

10. The system of claim 9, wherein the excitation light is an evanescent wave.

11. The system of claim 9, wherein a protein that interacts with a sugar chain is a lectin, an enzymatic protein having a sugar-binding domain, a cytokine having an affinity for a sugar chain, or an antibody that interacts with a sugar chain.
